(19) United States
(12) Patent Application Publication
(10) Pub. No.: US 2025/0223629 A1
(43) Pub. Date: Jul. 10, 2025

(54) SUBSTRATE-BASED PROTEIN ASSAY WITHOUT PROTEIN SUBSTRATE BINDING

(71) Applicant: Nautilus Subsidiary, Inc., Seattle, WA (US)

(72) Inventors: Ashwin Gopinath, Palo Alto, CA (US); Paul Rothemund, Pasadena, CA (US); Shane Bowen, Encinitas, CA (US); Rachel Galimidi, Encinitas, CA (US); Bhavik Nathwani, San Diego, CA (US)

(21) Appl. No.: 18/834,424

(22) PCT Filed: Jan. 31, 2023
(86) PCT No.: PCT/US2023/012033

§ 371 Date: Jul. 30, 2024

Related U.S. Application Data
(60) Provisional application No. 63/305,106, filed on Jan. 31, 2022.

(51) Int. Cl. C12Q 1/6804; G01N 15/1429

(52) U.S. Cl. CPC C12Q 1/6804; G01N 15/1429

(57) ABSTRACT

Provided herein in various embodiments, is a method for detecting and/or quantifying an analyte molecule present in a sample without employing a sequencing operation. As discussed, detection supramolecular structures are used to perform the detection and/or quantification of the analyte of interest. In one embodiment the detection supramolecular structures include a supramolecular structure (e.g., a nucleic acid origami structure) that comprises a core structure composed of one or more core molecules, a single affinity binder linked to the supramolecular structure at a first location, and one or more unique identifiers also attached to the supramolecular structure and which convey information about the affinity binder present on a respective detection supramolecular structure.

### BACKGROUND

The current state of personalized healthcare is generally genome-centric, focused on quantifying the genes present within an individual. While such an approach has proven to be extremely powerful, it does not provide a clinician with the complete picture of an individual's health. This is because, while genes are the “blueprints” of an individual, for many conditions they merely inform the likelihood of developing an ailment. Within an individual these “blueprints” first need to be transcribed into RNA and then translated into various protein molecules in order to have an effect on the health of an individual.

The concentration of proteins, the interaction between the proteins (protein-protein interactions or PPI), as well as the interaction between proteins and other molecules, are intricately linked to the health of different organs, homeostatic regulatory mechanism as well as the interaction of these systems with the external environment. Hence, identification of one or more proteins present within a patient sample (e.g., a blood sample or biopsy), as well as quantification (either absolute or relative) of such proteins, is useful to create a complete picture of an individual's health at a given time as well as to predict any emerging health issues. For instance, the amount of stress experienced by cardiac muscles (e.g., during a heart attack) can be inferred by measuring the concentration of troponin I/II and myosin light chain present within peripheral blood. Similar protein biomarkers have also been identified, validated, and are deployed for a wide variety of organ dysfunctions (e.g., liver disease and thyroid disorders), specific cancers (e.g., colorectal or prostate cancer), and infectious diseases (e.g., HIV and Zika). The interactions between these proteins are also useful for drug development and are a valuable dataset. The ability to detect and quantify proteins and protein interaction with other molecules within a given sample of bodily fluids is an integral component of such healthcare development.

### BRIEF DESCRIPTION OF THE DRAWINGS

Specific embodiments of the disclosed devices, delivery systems, or methods will now be described with reference to the drawings. Nothing in this detailed description is intended to imply that any particular component, feature, or step is essential to the invention.

FIG. 1 depicts a process flow illustrating steps in detecting the presence and/or quantity of an analyte of interest using detection supramolecular structures, in accordance with aspects of one implementation of the present technique;

FIG. 2 depicts a hydrogel matrix to which localization supramolecular structures are attached, in accordance with aspects of one implementation of the present technique;

FIG. 3 depicts the binding of analyte molecules to the localization supramolecular structures of FIG. 2 and the separation of unbound analyte, in accordance with aspects of one implementation of the present technique;

FIG. 4 depicts a detection supramolecular structure, with inset enlargements of an affinity binder linkage and a fluorescent molecule linkage, in accordance with aspects of one implementation of the present technique;

FIG. 5 depicts the binding of detection supramolecular structures to the localized analyte molecules of FIG. 3 and the separation of unbound detection supramolecular structures, in accordance with aspects of one implementation of the present technique;

FIG. 6 depicts the detachment of positive detection supramolecular structures from their respective affinity binders, in accordance with aspects of one implementation of the present technique;

FIG. 7 depicts the detachment and separation of positive detection supramolecular structures, in accordance with aspects of one implementation of the present technique;

FIG. 8 depicts binding of positive detection supramolecular structures to a detection substrate, in accordance with aspects of one implementation of the present technique;

FIGS. 9A, 9B, 9C, and 9D depict, respectively, various multiplexing detection techniques, in accordance with aspects of the present technique;

FIGS. 10A and 10B depict, respectively, additional multiplexing detection techniques, in accordance with aspects of the present technique;

FIG. 11 depicts a process flow illustrating steps in detecting the presence and/or quantity of an analyte of interest using detection supramolecular structures, in accordance with aspects of a further embodiment of the present technique;

FIG. 12 depicts a hydrogel matrix to which affinity binders are attached, in accordance with aspects of one implementation of the present technique;

FIG. 13 depicts the binding of analyte molecules to the localization affinity binders of FIG. 12 and the attachment of detection supramolecular structures, in accordance with aspects of one implementation of the present technique;

FIG. 14 depicts the binding of detection supramolecular structures to the localized analyte molecules of FIG. 13 and the separation of unbound detection supramolecular structures, in accordance with aspects of one implementation of the present technique;

FIG. 15 depicts the detachment of positive detection supramolecular structures from their respective affinity binders, in accordance with aspects of one implementation of the present technique;

FIG. 16 depicts the detachment and separation of positive detection supramolecular structures, in accordance with aspects of one implementation of the present technique;

FIG. 17 depicts a process flow illustrating steps in detecting the presence and/or quantity of an analyte of interest using detection supramolecular structures, in accordance with aspects of an additional embodiment of the present technique;

FIG. 18 depicts a bead structure to which localization affinity binders are attached, the binding of analyte molecules to the localization affinity binders, and the binding of detection supramolecular structures to the localized analyte molecules, in accordance with aspects of one implementation of the present technique;

FIG. 19 depicts the binding of detection supramolecular structures to the localized analyte molecules and the separation of unbound detection supramolecular structures, in accordance with aspects of one implementation of the present technique;

FIG. 20 depicts the detachment of positive detection supramolecular structures from their respective affinity binders, in accordance with aspects of one implementation of the present technique;

FIG. 21 depicts the detachment and separation of positive detection supramolecular structures, in accordance with aspects of one implementation of the present technique;

FIG. 22 depicts a process flow illustrating steps in detecting the presence and/or quantity of an analyte of interest using detection supramolecular structures, in accordance with aspects of an additional embodiment of the present technique;

FIG. 23 depicts a bead structure to which localization supramolecular structures are attached, the binding of analyte molecules to the localization supramolecular structures, and the separation of unbound analyte, in accordance with aspects of one implementation of the present technique;

FIG. 24 depicts the binding of detection supramolecular structures to the localized analyte molecules of FIG. 23 and the separation of unbound detection supramolecular structures, in accordance with aspects of one implementation of the present technique;

FIG. 25 depicts the detachment of positive detection supramolecular structures from their respective affinity binders, in accordance with aspects of one implementation of the present technique;

FIG. 26 depicts the detachment and separation of positive detection supramolecular structures, in accordance with aspects of one implementation of the present technique;

FIG. 27 shows a block diagram of an example processing system according to embodiments of the present disclosure;

FIG. 28 depicts an example of positive detection supramolecular structure placement on a chip substrate, in accordance with aspects of the present techniques;

FIG. 29 depicts a positive correlation between the number of observable binding site events on a chip substrate and the concentration of positive detection supramolecular structures in solution, in accordance with aspects of the present techniques;

FIG. 30 graphically depicts quantified assay results for interleukin-8, in accordance with aspects of the present techniques;

FIG. 31 graphically depicts quantified assay results for tumor necrosis factor alpha, in accordance with aspects of the present techniques; and

FIG. 32 graphically depicts quantified results for a multiplexed assay of interleukin-8 and tumor necrosis factor alpha having inversely varying concentration trends, in accordance with aspects of the present techniques.

### SUMMARY

The present disclosure generally relates to systems, structures and methods for detection and quantification of one or more analytes of interest (e.g., analyte molecules), such as a protein or proteins, that are present in a sample.

Provided herein in various embodiments, are methods for detecting and/or quantifying molecules of an analyte (or analytes), such as a protein or proteins of interest, present in a sample without employing a sequencing operation and without binding the analyte molecules to the detection substrate from which the quantification is performed. In certain embodiments, the analyte molecules are paired in a 1:1 manner with an intermediary structure or nanoparticle, such as a DNA origami, which is instead detected and/or counted at an assay step to correspondingly provide a count of the analyte molecules. In such an embodiment, the analyte molecules themselves may be absent from the detection and/or counting step (i.e., not present in the solution or bound to the detection substrate) though an exact count of the analyte molecules is still obtained due to the 1:1 correspondence with the nanoparticle intermediaries.

In one embodiment, and as described in greater detail below, such an assay operation includes the use of a detection supramolecular structure (e.g., a nucleic acid origami structure) that comprises a core structure composed of one or more core molecules. The detection supramolecular structure is also linked to an affinity binder (e.g., a single affinity binder) at a first location and an anchor or barcode strand or molecule (e.g., a single stranded nucleic acid strand, such as a single stranded RNA or DNA molecule) at a second location. In a further embodiment, one or more different types of fluorophores are also attached to the detection supramolecular structure and convey, when active or excited, information about the affinity binder present on the detection supramolecular structure.

By way of example, the one or more types of fluorophores may, alone or taken in the aggregate, function as an identifier of the affinity binder and may incorporate different fluorescent molecules that emit, when excited, at a known frequency or frequency range and/or that emit only when excited by radiation (e.g., light) at a known frequency or frequency range). As described herein such an identification function may be understood to provide unique identifying information that may be used to identify or characterize a supramolecular structure as having a particular affinity binder attached (or previously attached), i.e., as being specific to a respective analyte molecule. By way of example, in a single unique identifier context a known frequency of emission associated with a unique identifier may correspond to a known affinity binder, and hence a known analyte. In other contexts where signal multiplexing is contemplated, different combinations of emitted frequencies and/or proportions of emitted frequencies may be associated with different respective affinity binders, thereby allowing multiple analytes to be detected and/or counted during a given assay operation. In such multiplexing contexts, data generation and collection may be multi-channel in that emission data is generated and collected using multiple readout channels, each corresponding to a different emission spectrum. In practice, this may take the form of one readout channel per unique or identifying sequence or emission frequency or spectrum employed.

As discussed herein, the detection supramolecular structures may be contacted with a sample that potentially contains molecule of the analyte or analytes (e.g., protein or proteins) of interest, such as in a solution phase. In certain embodiments, as discussed herein, the analyte(s) of interest may, before exposure to the detection supramolecular structure, be exposed to or contacted with an initial binding or localization medium, examples of which include, but are not limited to: additional supramolecular structures (e.g., localization supramolecular structures) including other affinity binders for the analyte(s) of interest and conjugated with a hydrogel matrix; other affinity binders for the analyte(s) of interest bound or linked directly to a hydrogel matrix; other affinity binders for the analyte(s) of interest bound or linked to a bead structure (e.g., magnetic beads); or additional supramolecular structures (e.g., localization supramolecular structures) including other affinity binders for the analyte(s) of interest and conjugated with such bead structures.

In such contexts, the analyte of interest may be initially bound or complexed with a localizing affinity binder, which, as noted above, may be associated with a structure such as a hydrogel matrix, bead structure, or other suitable structure, such as a localizing supramolecular structure bound to a hydrogel matrix or magnetic bead. The detection supramolecular structures (e.g., detector origami structures) having the relevant affinity binders may be contacted with (e.g., mixed in solution, flowed through a flow channel (e.g., capillary tube) having a hydrogel matrix within, etc.) the bound or complexed analyte(s) so as to bind with the bound or complexed analytes at a different site on the respective analyte. In such a context, unbound detection supramolecular structure (e.g., detector origami) may then be washed away, along with any remaining unbound protein.

In certain such embodiments, the detection supramolecular structures (e.g., detector origami structures) may be released from the analyte of interest via a chemical or photocleavage operation, displacement (e.g., strand displacement) operation, or other suitable attachment breaking operation. The released detection supramolecular structures may be isolated, such as by washing, flushing, or otherwise separating the detection supramolecular structures from the binding complexed structures. In certain implementations, the isolated detection supramolecular structures may be hybridized to a detection substrate configured to allow counting of the hybridized detection supramolecular structures so as to provide a quantified assay result for the analyte of interest. As may be noted, in certain such embodiments the analyte may be absent at the detection hybridization stage such that the quantification is performed on the intermediary of the detection supramolecular structure, with no analyte being bound directly or indirectly to the detection substrate. Alternatively, in place of a detection substrate for binding of the detection supramolecular structures, the detection supramolecular structures may instead be counted in a solution phase, such as using flow cytometry. It should be appreciated, however, that the preceding explanation and example are provided by way of non-limiting illustration only and that various techniques for detecting and/or quantifying the detection supramolecular structure are contemplated and encompassed by the present discussion.

As discussed herein, detection supramolecular structures may be used in various techniques for detecting an analyte molecule of interest when the analyte molecule is present in the sample at a count of a single molecule or higher. Further, with respect to examples and explanations as discussed herein, a respective sample may comprise a complex biological sample and the described methodologies may increase the dynamic range of a detection operation and/or facilitate quantitative assessment of a range of molecular concentrations within the complex biological sample.

By way of example, as used herein a sample may comprise a biological sample, such as an aqueous solution comprising a protein, a peptide, a fragment of a peptide, a lipid, DNA, RNA, an organic molecule, a viral particle, an exosome, an organelle, or any complexes thereof. In some embodiments, the sample comprises or is derived from a tissue biopsy, blood, blood plasma, urine, saliva, tears, cerebrospinal fluid, extracellular fluid, cultures cells, culture media, discarded tissue, plant matter, synthetic proteins, prions, a bacterial and/or viral sample or fungal tissue, or combinations thereof. The sample may be isolated from a primary source such as cells, tissue, bodily fluids (e.g., blood), environmental samples, or combinations thereof, with or without purification. In embodiments where cells are involved in sample preparation the cells may be lysed using a mechanical process or other cell lysis methods (e.g., lysis buffer). The sample may be filtered using a mechanical process (e.g., centrifugation), micron filtration, chromatography columns, other filtration methods, or combinations thereof. Further, the sample may or may not be treated with one or more enzymes to remove one or more nucleic acids or one or more proteins. In certain implementations the sample is collected from one or more individual persons, one or more animals, one or more plants, or combinations thereof. By way of example, the sample may be collected from an individual person (e.g., a patient of subject), animal and/or plant having a disease or disorder that comprises an infectious disease, an immune disorder, a cancer, a genetic disease, a degenerative disease, a lifestyle disease, an injury, a rare disease, an age-related disease, or combinations thereof.

The sample may further be an environmental sample, such as a wastewater or soil sample. Further, the sample may also be a non-biological sample. In an embodiment, the sample may be a sample from a chemical process step, a sample of food or nutritional components, or packaging components. In some embodiments, the analyte molecule of interest within a given sample may comprise a protein, a peptide, a peptide fragment, a lipid, a DNA, a RNA, an organic molecule, an inorganic molecule, complexes thereof, or any combinations thereof.

In some embodiments, each core structure of a respective supramolecular structure, whether a detector supramolecular structure or a localization detector supramolecular structure as discussed herein, is a nanostructure. In certain implementations, each core structure of a plurality of supramolecular structures (e.g., a plurality of detector origami structures) are identical to each other. In some embodiments, each supramolecular structure (such as detector origami structures or localization origami structures) comprises a prescribed shape, size, molecular weight, or combinations thereof, so as to reduce or eliminate cross-reactions when multiple supramolecular structures, of the same or different types, are present. In some embodiments, the one or more core molecules for each core structure comprises one or more nucleic acid strands, one or more branched nucleic acids, one or more peptides, one or more small molecules, or combinations thereof. By way of example, each core structure may independently comprises a deoxyribonucleic acid (DNA) origami (e.g., a scaffolded DNA origami), a ribonucleic acid (RNA) origami (e.g., a scaffolded RNA origami), a hybrid DNA: RNA origami (e.g., a scaffolded hybrid (DNA: RNA) origami), a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

In certain implementations the respective analyte molecule is bound to the respective affinity binder or reactive group of a localization supramolecular structure or and/or detection supramolecular structure through a chemical bond. In some embodiments, the affinity binder or reactive group comprises a protein, a peptide, an antibody, an aptamer (e.g., RNA and DNA), a darpin, a polymer like PEG, or combinations thereof. In some embodiments, for each supramolecular structure (detection or localization) the affinity binder or reactive group is linked to the core structure of the supramolecular structure via complementary binding. By way of example, an affinity binder linker structure of the respective affinity binder may form a bond with a complementary linker structure bound or otherwise attached to the respective supramolecular structure.

In some embodiments, each supramolecular structure (detection or localization) may further comprise an anchor molecule linked to the core structure. In some embodiments, the anchor molecule comprises an amine, a thiol, a DBCO, a maleimide, biotin, an azide, an acrydite, an NHS-ester, a single stranded nucleic acid (RNA or DNA) of specific sequence, or combinations thereof. In such embodiments, the anchor molecule may facilitate binding of the supramolecular structure to respective binding sites on a surface of a substrate, such as to an attachment site of a hydrogel matrix or magnetic bead (in the context of a localization supramolecular structure) or to an attachment site or surface of a detection substrate (in the context of a detection supramolecular structure). Such surfaces or substrates may comprise a plurality of binding sites, wherein each binding site is configured to link with a corresponding supramolecular structure via the respective anchor molecule. By way of example, when bound to the surface of a detection substrate, a detection supramolecular structure may undergo a readout process, such as a series of excitation and emission steps whereby detection supramolecular structures attached to the detection substrate are interrogated to classify and/or quantify an analyte molecule. Alternatively, as described herein the detection supramolecular structures described herein may be read-out or otherwise detected and quantified while in solution (i.e., not bound to a substrate), such as using flow cytometry.

### DETAILED DESCRIPTION

Disclosed herein are structures and methods for detecting one or more types of analyte molecules present in a sample. In some embodiments, information (e.g., presence, counts, and so forth) about the analyte molecules within a given sample is determined subsequent to binding of a detection supramolecular structure to an analyte molecule, either in solution or in a context where the analyte molecules have been fixed or localized to a substrate or structure. As used herein, each supramolecular structure (detection or localization) includes as part of its structure a single affinity binder that is specific to a respective analyte molecule. In certain embodiments each detection supramolecular structure may also include one or more unique identifiers (e.g., “barcodes”) such that the barcode or combination of barcodes (e.g., the respective barcodes that are present and/or the ratio of respective barcodes that are present) is unique to detection supramolecular structures based on their respective analyte affinity (i.e., the attached affinity binder). The barcodes may then be read-out by a detection apparatus as discussed herein to classify and/or quantify analyte molecules of interest present in the sample.

As discussed in greater detail below, supramolecular structures include a core structure composed of one or more core molecules. By way of example, the core structure may be a nucleic acid origami structure, such as a DNA origami structure. As discussed herein, in certain embodiments a single affinity binder and/or one or more unique identifier(s) are attached (e.g., linked) to the supramolecular structure to facilitate a detection or a localization functionality.

As discussed herein, detection supramolecular structures bound or previously bound to an analyte molecule may be detected and/or counted by a suitable read-out or detection mechanism. In general, any suitable detection mechanism may be employed, including detection mechanisms based on optical, electrical, or magnetic detection schemes. For example, detection supramolecular structures may remain free in a solution phase subsequent to exposure to a sample, with read-out and detection being performed while the detection supramolecular structure is free in the solution phase, such as via flow cytometry. Alternatively, in certain embodiments the detection supramolecular structures may be linked to or immobilized on a substrate after sample exposure, with readout occurring while the detection supramolecular structures are immobilized on the substrate.

Thus, as provided herein, an analyte molecule can be associated with an individual detection supramolecular structure which is then separately counted or detected to generate detection and/or quantification results. Within a given detection and/or quantification operation a sample may be processed using a variety of sets of detection supramolecular structures, each set having a different affinity binder, such that each set of detection supramolecular structures has an affinity for a different analyte molecule of interest. Such an approach allows a sample having an uncharacterized composition of multiple possible analytes of interest to analyzed and characterized for the presence and/or concentration of multiple particular analytes of interest. For example, a human sample can be characterized to determine a presence and/or concentration of one or more proteins, peptides, peptide fragments, lipids, nucleic acids, organic molecules, inorganic molecules, and so forth, of interest. Alternatively, as may be appreciated, if only a single analyte molecule is of interest, only detection supramolecular structures having respective affinity binders specific to the particular analyte molecule of interest, may be used to process the sample.

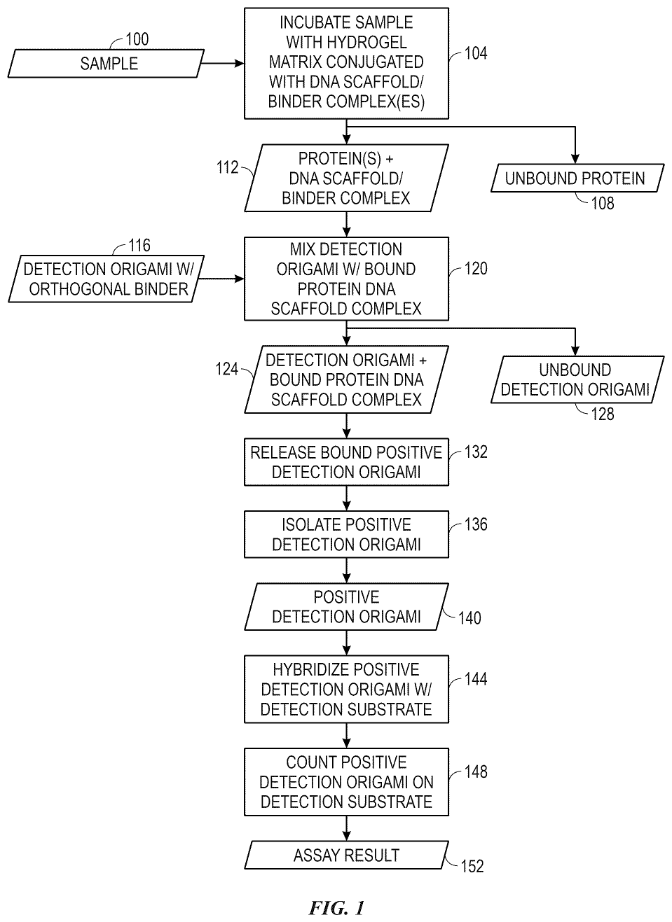

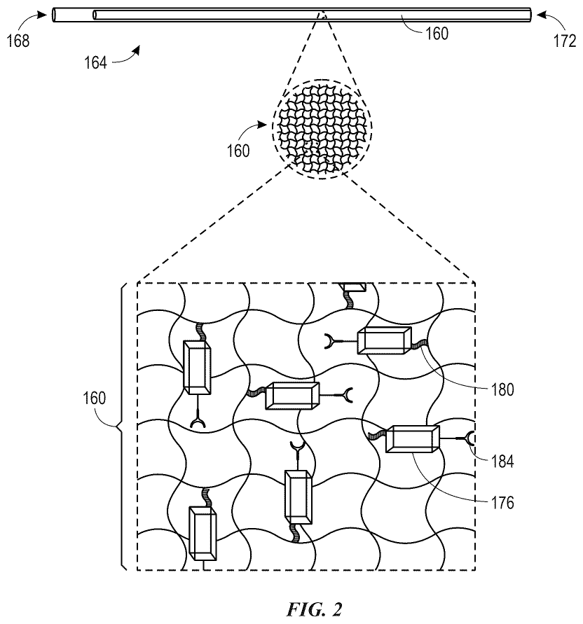

With the preceding in mind, examples of implementations of the present techniques are provided in greater detail below. By way of example, and turning to FIG. 1, a process flow of an assay operation utilizing detection supramolecular structures is depicted. FIGS. 2-9 depict various aspects of the process flow of FIG. 1 so as to facilitate visualization and explanation of the described process flow. Detection and localization supramolecular structures as discussed herein may encompass a supramolecular structures or core structures on which other relevant molecular structures are attached. As described herein, detection supramolecular structures may be understood to be supramolecular structures which are detected and/or counted as part of an assay or detection step. Conversely, localization supramolecular structures may localize or bind an analyte molecule present in a sample to a substrate or surface prior to exposure to the detection supramolecular structures.

In some embodiments, a respective detection or localization supramolecular structure is a programmable structure that can spatially organize molecules. Further, in certain implementations the supramolecular structure comprises a plurality of molecules linked together, some or all of which may interact with one another.

Additionally, a respective supramolecular structure may have a specific shape or geometry, e.g., a substantially planar shape that has a longest dimension in an x-y plane. In some embodiments, a supramolecular structure is a nanostructure, such as a nanostructure that comprises a prescribed molecular weight based on the plurality of molecules forming the supramolecular structure. The plurality of molecules may, for example, be linked together through a bond, a chemical bond, a physical attachment, or combinations thereof. In certain implementations the supramolecular structure comprises a large molecular entity, of specific shape and molecular weight, formed from a well-defined number of smaller molecules interacting specifically with each other. The structural, chemical, and physical properties of the supramolecular structure may be explicitly designed. By way of example, the supramolecular structure may comprise a plurality of subcomponents that are spaced apart according to a prescribed distance. In some embodiments, at least a portion of the supramolecular structure (or its constituent core structure) is rigid or semi-rigid. Correspondingly or alternatively, all or parts of the supramolecular structure (or its constituent core structure) may be flexible or conformable. In certain embodiments the supramolecular structure is at least 50 nm-200 nm in at least one dimension. In certain embodiments the supramolecular structure is at least 20 nm long in any dimension.

In general, a supramolecular structure as described herein may comprise a core structure which may be a polynucleotide structure, a protein structure, a polymer structure, or a combination thereof. In some embodiments, the core structure comprises either one core molecule or two or more core molecules linked together. By way of example, the one or more core molecules may comprise 2, 3, 4, 5, 6, 7, 8, 9, 10, 20, 50, 100, 200 or 500 unique molecules that are linked together. In some embodiments, the one or more core molecules comprises from about 2 unique molecules to about 1,000 unique molecules. In certain implementations, the one or more core molecules interact with each other and define the specific shape of the respective supramolecular structure. By way of example, the plurality of core molecules may interact with each other through reversible non-covalent interactions.

In some embodiments, the specific shape of the core structure of a supramolecular structure has a three-dimensional (3D) configuration. Further, the one or more core molecules may provide a specific molecular weight. For example, all core structures of a plurality of supramolecular structures may have a same configuration, size, and/or weight, but may differ in their attached linker sequences and/or other attached molecules, as described herein. However, excluding such differing linkers or other attached molecules, the supramolecular structures of such a plurality may be otherwise identical. In certain examples the core structure may be a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the core structure comprises an entirely polynucleotide structure.

In some embodiments, the supramolecular structure or its constituent core structure(s)) comprise a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami with multiple scaffolds, a peptide structure, an enzymatically synthesized nucleic acid structure (e.g., nanoball(s)), or combinations thereof. As discussed herein, in such embodiments the DNA origami, RNA origami, or hybrid DNA/RNA origami may be scaffolded. As used herein, the term “scaffold” or “scaffolded” refers to the use or inclusion of a circular ssDNA molecule, called a “scaffold” strand, that is folded into a predefined 2D or 3D shape by interacting with two or more short ssDNA, called “staple” strands, which interact with specific sub-sections of the ssDNA “scaffold” strand. In some embodiments, the core structure comprising a DNA origami, RNA origami, or hybrid DNA/RNA origami has a prescribed two-dimensional (2D) or 3D shape.

In an example embodiment, the core structure(s) of a supramolecular structure may be a nucleic acid origami that has at least one lateral dimension between about 20 nm to about 1 μm. In an embodiment, the nucleic acid origami has at least one lateral dimension between about 20 nm to about 200 nm, about 20 nm to about 400 nm, about 20 nm to about 600 nm, about 20 nm to about 800 nm, about 100 nm to about 200 nm, about 100 nm to about 300 nm, about 100 nm to about 400 nm, about 100 nm to about 500 nm, about 200 nm to about 400 nm by way of example. Further, in certain embodiments the nucleic acid origami has at least a first lateral dimension between about 20 nm to about 1 μm and a second lateral dimension, orthogonal to the first, between about 20 nm to about 1 μm. In one implementation the nucleic acid origami has a planar footprint having an area of about 200 nm² to about 1 μm².

In some embodiments, some or all of the components (e.g., constituent components) of the supramolecular structure may be independently modified or tuned. By way of example, modifying one or more of the components of the supramolecular structure may modify the 2D and 3D geometry of the supramolecular structure itself. In some embodiments, modifying one or more of the components of the supramolecular structure may modify the 2D and 3D geometry of the core structure of the supramolecular structure. In some embodiments, such capability for independently modifying the components of the supramolecular nanostructure enables precise control over the organization, geometry, or other steric or functional properties of one or more supramolecular structures.

With the preceding high-level discussion of supramolecular structures, as used herein, in mind, an example a synthesis operation for a suitable supramolecular structure is provided. In this example, the synthesized supramolecular structure may be a scaffolded DNA origami. In such an example a scaffold (e.g., a circular ssDNA molecule of known sequence, which may be referred to as a “scaffold” strand) may be combined with a plurality of “staples” (e.g., two or more short ssDNA, called “staple” strands, which interact with specific sub-sections of the ssDNA “scaffold” strand). The staples selectively bind to specified locations on the scaffold such that a self-assembly of the supramolecular structure, e.g., a DNA origami in this example, is performed. In particular, the self-assembly step results in the scaffold being folded into a predefined 2D or 3D shape via interactions with the staples. In one embodiment, the staples are formed with excess thymine (T) located at nicks as well as crossovers so as to facilitate the crosslinking (i.e., formation of covalent crosslinks) of the staple structures forming the DNA origami when exposed to an energy source (e.g., UV illumination). Such cross-linking may help improve the thermostability of the formed DNA origami. In practice, the cross-linking step may be performed after the DNA origami is purified away from any unattached staple strands.

With the preceding background and context in mind, and turning to the process flow of FIG. 1, in the depicted process flow of one implementation, a sample 100 containing one or more analytes of interest, is incubated (step 104) with a complex of supramolecular structures (e.g., localization supramolecular structures, such as localization DNA origamis) and scaffold structures (e.g., a hydrogel matrix), which may jointly be referred to, in one such embodiment, as a DNA scaffold-binder complex.

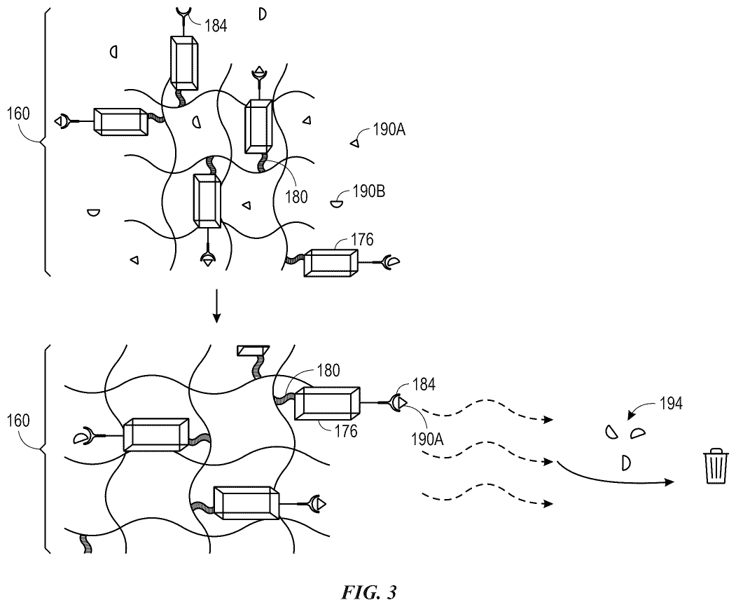

With this in mind, and turning to FIGS. 2 and 3, possible implementations are illustrated to better illustrate aspects of this step. Turning to FIG. 2, a hydrogel matrix 160 is illustrated as being disposed within a flow channel 164 (e.g., a capillary tube or channel within a microfluidic device or chip) having an input port 168 and an output port 172 through which the sample may be flowed. By way of example a hydrogel, as used herein, may be a polyacrylamide, polyimide, or other suitable matrix). As shown in the successive expanded views of FIG. 2, The hydrogel matrix 160 may be complexed with localization supramolecular structures 176 (e.g., nucleic acid origami structures having a core origami structure with anchor molecule 180 and affinity binder 184 attached constituents).

In some embodiments, each localization supramolecular structure 176 comprises an anchor molecule 180 linked to the core structure. In some embodiments, the anchor molecule 180 comprises an amine, a thiol, a DBCO, a maleimide, biotin, an azide, an acrydite, an NHS-ester, a single stranded nucleic acid (RNA or DNA) of specific sequence, or combinations thereof. In such embodiments, the anchor molecule 180 may facilitate binding of the localization supramolecular structure 176 to respective binding sites on a surface of a substrate, such as to an attachment site of a hydrogel matrix or bead structure. Such surfaces or substrates may comprise a plurality of binding sites, wherein each binding site is configured to link with a corresponding localization supramolecular structure via the respective anchor molecule 180. By way of example, when bound to a hydrogel matrix 160, the localization supramolecular structure 176, and any analyte bound to the localization supramolecular structure 176 may be substantially fixed in location with respect to the hydrogel matrix 160. By way of example, in the depicted example, each localization supramolecular structure 176 has an anchor molecule 180 in the form of a single strand nucleic acid anchor strand that is complementary to a complement strand of nucleic acid seeded on the hydrogel matrix 160.

Each localization supramolecular structure 176 also includes, bound to the core structure, an affinity binder 184 (or reactive group) that is specific to the analyte molecule of interest, such as to a specific binding region or domain on the analyte molecule of interest. As discussed herein, a respective analyte molecule may bind with specificity to the affinity binder 184 or reactive group of a localization supramolecular structure 176 through a chemical bond. In some embodiments, the affinity binder 184 or reactive group comprises a protein, a peptide, an antibody, an aptamer (e.g., RNA and DNA-based aptamers), a darpin, a polymer like PEG, or combinations thereof.

In certain embodiments attachment of the affinity binder 184 is via a linker structure or strand that chemically links to random or targeted (i.e., non-random) locations (e.g., complementary linker structures or strands) on one or more core molecules of the core structure of the localization supramolecular structure 176. By way of example, and in the context of affinity binders 184 (e.g., antibodies) linked to a DNA origami structure, such linkage may be accomplished using complementary nucleic acid strand pairing or other complementary pairing, via covalent or other chemical bond formation, and/or by any other suitable attachment mechanism. By way of example, linkage of affinity binders 184 may be accomplished via the mechanism of linking to specific and known “staple” strands of nucleic acid used in the formation of scaffolded nucleic acid origami structures. In such an embodiment, because the sequence of such staple strands is known and because they occur in a fixed quantity and at fixed locations on the origami structure, the affinity binder 184 may be positioned on the localization supramolecular structure 176 at a known and specific location. By way of example, certain of the staples may react with or link to a respective polymer linker with specificity, where the polymer linker corresponds to placement of the affinity binder 184. Due to the selectivity of the staples in terms of binding to specific locations on the scaffold, an affinity binder 184 may be targeted for attachment to a respective staple which selectively binds to the scaffold at a known and specific location on the localization supramolecular structure 176 (e.g., DNA origami). The resulting localization supramolecular structure 176 has binding specificity to a specific analyte molecule, as determined by the affinity binder 184, and in the presence of the specific analyte molecule will bind to one such molecule.

In the depicted example of FIG. 2, it may be appreciated that the localization supramolecular structure 176, when binding an analyte molecule of interest via affinity binder 184, thereby localizes the bound analyte of interest to the substrate (here the hydrogel matrix 160) for subsequent operations, as discussed herein. Turning to FIG. 3, which illustrates aspects of the step 104, a sample comprising analytes 190, including analytes of interest 190A, is exposed to the hydrogel matrix 160 in this embodiment. All or some portion of the analyte molecules of interest 190A bind to the affinity binders 184 present on the localization supramolecular structures 176 bound to the hydrogel matrix 160. By way of example, the analyte molecules of interest may bind via a specific interaction to the affinity binders 184 due to the specificity of the affinity binder 184 for the analyte of interest 190A. Some portion of other analyte molecules (i.e., analytes not of interest 190B) may also interact with the affinity binders 184 via non-specific interactions, but such interactions, if present will be weak and limited in number.

After an incubation period, the unbound analyte molecules 194 may be flowed or washed away through the flow channel 164 so as to remove unbound analyte molecules, such as analytes not of interest 190B, as shown in the bottom step illustrated in FIG. 3. At this stage, therefore, primarily analytes of interest 190A remain bound to the substrate (e.g., hydrogel matrix 160), with unbound analytes having been removed via the output port 172 of the flow channel 164.

Turning back to FIG. 1, this is illustrated in the example, process flow in the context of a protein assay in which a nucleic acid origami forms the basis of the localization supramolecular structures 176. Hence unbound protein 108 is shown as being separated and removed subsequent to the incubation step 104, leaving the protein of interest bound to the DNA scaffold-binder complex (complex 112 in the aggregate).

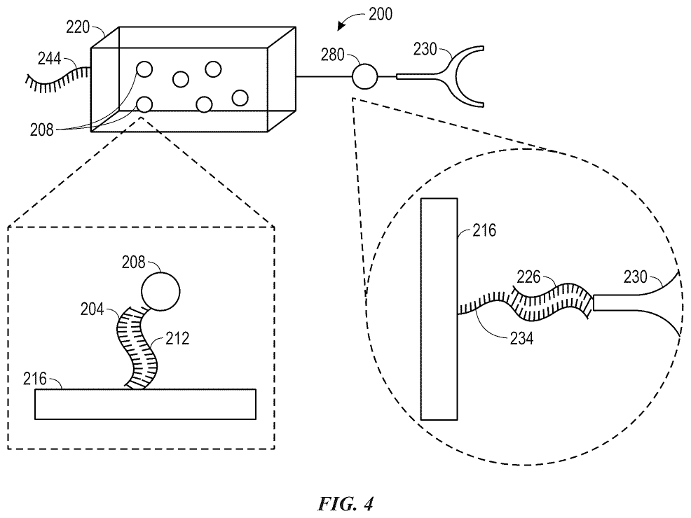

As illustrated in FIG. 1, at a next stage a detection supramolecular structure (e.g., a detection origami 116 in the example of FIG. 1) is mixed or flowed over the protein scaffold/binder complex 112, such as via flowing through the flow channel 164. Turning to FIG. 4, an example of one such detection supramolecular structure 200 is shown in the context of DNA origami with bound anchor and affinity binder constituents. As shown in the example of FIG. 4, and as noted above, supramolecular structures (detection and/or localization) as used herein may be configured to bind to a single analyte molecule of interest (or a specific region or domain of such an analyte) due to a single affinity binder being linked to the supramolecular structure. The presence of a single affinity binder on each detection supramolecular structure, as may be appreciated, may correspond to a single molecule of the analyte of interest, hence providing a 1:1 conversion ratio of analyte to detection supramolecular structures. Such a single analyte molecule binding event might typically be associated with a relatively small or low signal strength. In the present context, so that the detection supramolecular structure 200 is a strong signal source during a subsequent detection step, the detection supramolecular structure 200 as used herein may be bound to one or more types (e.g., 1 to 8) of unique identifier, with each type of unique identifier being present in multiple copies (e.g., tens, hundreds, thousands, and so forth) on the detection supramolecular structure 200.

As shown in FIG. 4, and in the context of a detection supramolecular structure based on DNA origami, in one embodiment each type of unique identifier comprise a respective fluorescent molecule 208, such as a fluorescent molecule 208 attached to a linker structure or strand 212 that chemically links to random or targeted (i.e., non-random) locations (e.g., complementary linker structures or strands 204) on one or more core molecules 216 of the core structure 220 of the detection supramolecular structure 200. By way of example, fluorescent molecules 208 as used herein may encompass, but are not limited to, dye molecules, quantum dots, or polymers.

By way of example, and in the context of fluorescent molecules linked to a DNA origami structure, such linkage may be accomplished using complementary nucleic acid strand pairing or other complementary pairing, via covalent or other chemical bond formation, and/or by any other suitable attachment mechanism. By way of example, linkage of fluorescent molecules 208 may be accomplished via the mechanism of linking to specific and known “staple” strands of nucleic acid used in the formation of scaffolded nucleic acid origami structures. In such an embodiment, because the sequence of such staple strands is known and because they occur in a fixed quantity and at fixed locations on the origami structure, the same or different types of fluorescent molecules 208 may be positioned on the detection supramolecular structure 200 at known and specific locations and numbers. By way of example, certain of the staples may react with or link to polymer linkers with specificity, where the polymer linkers correspond to fluorescent molecules 208. Due to the selectivity of the staples in terms of binding to specific locations on the scaffold, fluorescent molecules 208 may be targeted for attachment to respective staples which selectively bind to the scaffold at spatially separated locations to ensure the separation of the fluorescent molecules 208 on the detection supramolecular structure (e.g., DNA origami).

Each type of unique identifier (e.g., fluorescent molecules 208) may have a characteristic frequency at which it is excited or stimulated to emit detectable radiation (e.g., light) and/or a characteristic frequency at which it emits detectable radiation when excited. Detection supramolecular structures 200 having a particular analyte affinity (i.e., an attached affinity binder for a respective analyte molecule, as discussed below) may therefore have a characteristic identification sequence attached directly or indirectly (e.g., red, blue, yellow, or green, and so forth), a characteristic combination of fluorescent markers (each corresponding to a different identification sequence) attached (e.g., red+blue, yellow+green, red+yellow+blue, and so forth), and/or a characteristic ratio of fluorescent markers attached (e.g., (2 red: 1 blue), (3 red: 2 green: 1 yellow). In this manner, detection at a read-out step of a characteristic frequency, combination of frequencies, or ratio of frequencies) may be used to determine and/or count the presence of detection supramolecular structures 200 bound to a respective analyte molecule.

Alternatively, as discussed herein, specificity may be in the subsequent interaction between the detection supramolecular structure 200 and the detection substrate discussed below. In which case no particular specificity or identification based on the fluorophore 208 (or combination of fluorophores 208) may be required as any detectable signal (e.g., measurable fluorescence) on the detection substrate (or specific locations on the detection substrate) may be used to detect the present and/or quantity of the analyte of interest in the sample. In such an example, in a multiplexed signal context where attachment of the detection supramolecular structures is not random but instead targeted or controlled, binding location in combination with detectable signal may be used to detect and/or quantify different analytes of interest. Further, as noted herein, detection and/or quantification may, in other embodiments, be performed in a solution phase, with no binding to a detection substrate, such as via flow cytometry.

In addition, as shown in FIG. 4, an affinity binder (or reactive group) 230 may be attached to the detection supramolecular structure 200. As discussed herein, a respective analyte molecule may bind with specificity to the affinity binder 230 or reactive group of a detection supramolecular structure 200 through a chemical bond. In some embodiments, the affinity binder 230 or reactive group comprises a protein, a peptide, an antibody, an aptamer (e.g., RNA and DNA-based aptamers), a darpin, a polymer like PEG, or combinations thereof.

In the depicted example, attachment of the affinity binder 230 is via a linker structure or strand 226 that chemically links to random or targeted (i.e., non-random) locations (e.g., complementary linker structures or strands 234) on one or more core molecules 216 of the core structure 220 of the detection supramolecular structure 200. By way of example, and in the context of affinity binders 230 (e.g., antibodies) linked to a DNA origami structure, such linkage may be accomplished using complementary nucleic acid strand pairing or other complementary pairing, via covalent or other chemical bond formation, and/or by any other suitable attachment mechanism. By way of example, linkage of affinity binders 230 may be accomplished via the mechanism of linking to specific and known “staple” strands of nucleic acid used in the formation of scaffolded nucleic acid origami structures. In such an embodiment, because the sequence of such staple strands is known and because they occur in a fixed quantity and at fixed locations on the origami structure, the affinity binder 230 may be positioned on the detection supramolecular structure 200 at a known and specific location. By way of example, certain of the staples may react with or link to a respective polymer linker with specificity, where the polymer linker corresponds to placement of the affinity binder 230. Due to the selectivity of the staples in terms of binding to specific locations on the scaffold, an affinity binder 230 may be targeted for attachment to a respective staple which selectively binds to the scaffold at a known and specific location on the detection supramolecular structure 200 (e.g., DNA origami). The resulting detection supramolecular structure 200 has binding specificity to a specific analyte molecule, as determined by the affinity binder 230, and in the presence of the specific analyte molecule will bind to one such molecule.

In the context of a detection supramolecular structure 200, in certain embodiments the attachment of the affinity binder 230 to the core structure 220 is designed or configured to be broken. This is depicted in FIG. 4 as a detachment site 280 (e.g., cleavage or displacement site) at which the link between the core structure 220 and affinity binder 230 is broken, allowing separation of these two components. By way of example, detachment of the core structure 220 and affinity binder 230 may be accomplished by photo-cleavage, chemical cleavage, strand displacement (e.g., introduction of a complementary strand or strands that interfere with and displace the stand pairing between strands 226 and 234), or any other suitable detachment mechanism.

In addition, as illustrated in FIG. 4, each detection supramolecular structure 200 may further comprise an anchor molecule 244 linked to the core structure 220. In some embodiments, the anchor molecule 244 comprises an amine, a thiol, a DBCO, a maleimide, biotin, an azide, an acrydite, an NHS-ester, a single stranded nucleic acid (RNA or DNA) of specific sequence, or combinations thereof. By way of example, an anchor molecule 244 may form a bond with a complementary linker structure bound (e.g., complementary nucleic acid strand) or otherwise attached to a respective substrate. Further, in embodiments in which the anchor molecule 244 comprises a nucleic acid strand of a specific sequence, the anchor strand 244 may also serve as a unique identifier of the detection supramolecular structure 200, effectively acting as a unique identifier or barcode sequence for identification purposes.

As discussed herein, the anchor molecule 244 may facilitate binding of the detection supramolecular structure 200 (either random or targeted binding) to respective binding sites on an attachment site or surface of a detection substrate. Such surfaces or substrates may comprise a plurality of binding sites, wherein each binding site is configured to link with a corresponding detection supramolecular structure 200 via the respective anchor molecule 244. By way of example, when bound to the surface of a detection substrate, a detection supramolecular structure 200 may undergo a readout process, such as a series of excitation and emission steps whereby detection supramolecular structures 200 attached to the detection substrate are interrogated to classify and/or quantify an analyte molecule.

Turning back to FIG. 1, detection supramolecular structures 200, in the form of detection origami 116 in the depicted example, are flowed through the flow channel 164 and hydrogel matrix 160 so as to mix (step 120) with the protein scaffold/binder complex 112 formed by the localization supramolecular structures 176 and bound analyte 190A as shown in FIGS. 2 and 3. In the depicted example, the localization super molecular structures 176 and the detection supramolecular structures 200 have respective affinity binders 184, 230 that are specific to the analyte of interest 190A at different regions or domains (i.e., orthogonal binders). In this manner, both the localization supramolecular structure 176 and the detection supramolecular structure 200 can simultaneously bind to a respective analyte of interest 190A.

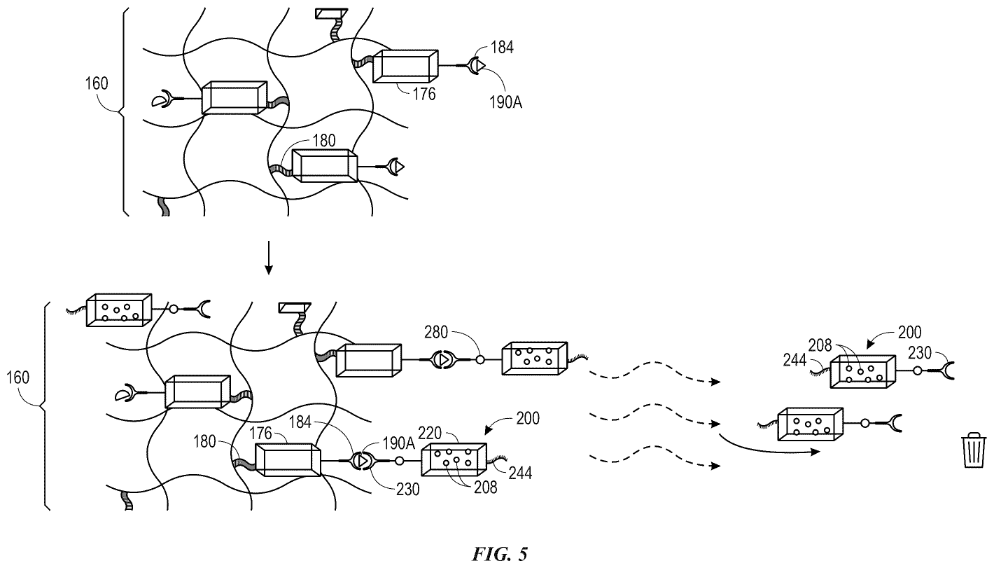

This is further illustrated in FIG. 5, in which the topmost illustration illustrates the protein scaffold/binder complex formed by the analyte of interest 190A and the localization supramolecular structures 176 prior to addition of the detection supramolecular structures 200 (top) and after addition of the detection supramolecular structures 200 (bottom). As shown in this illustration, analytes of interest 190A bound to the localization supramolecular structures 176 at this stage are also bound at different regions or domains to the detection supramolecular structures 200, such as via a different and distinct specific interaction. As also illustrated, at this stage, unbound detection supramolecular structures 200 may be washed away (e.g., flowed through the flow channel 164). Turning back to FIG. 1, in the detector origami implementation depicted, these outcomes are illustrated respectively by the unbound detection origami 128 that are washed away and the remaining detection origami/bound protein complex 124 that remain for further processing.

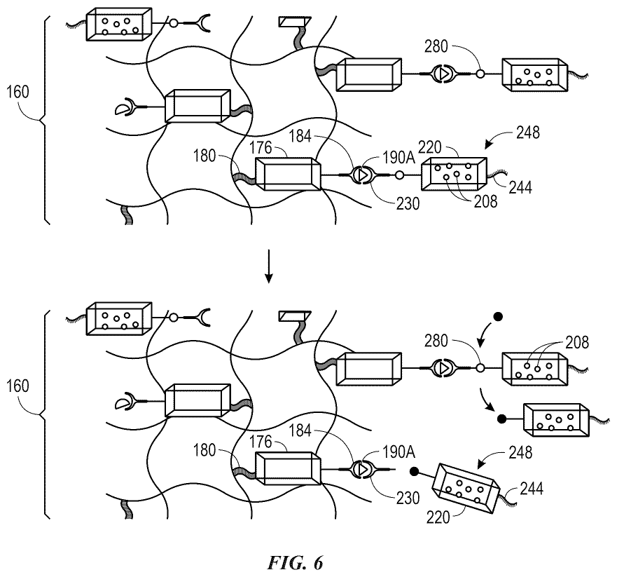

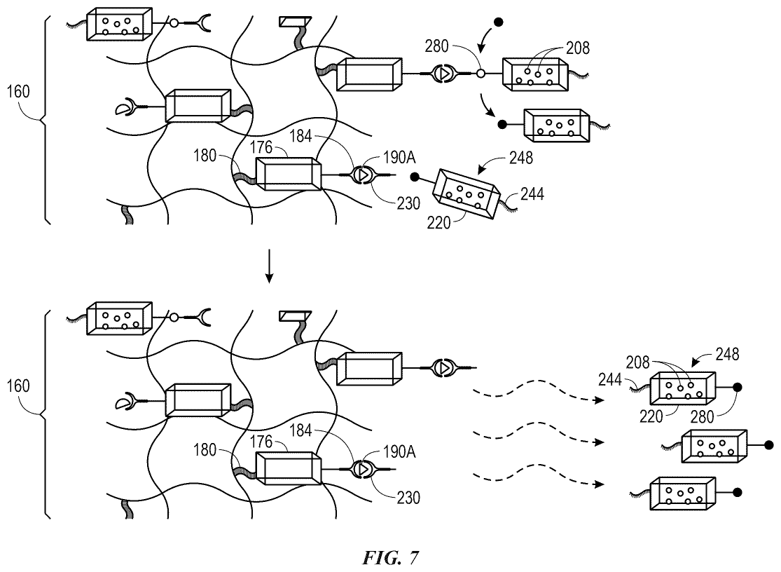

As further shown in FIG. 1, in a next step the detection origami that were successfully bound to analyte of interest at step 120, referred to as positive detection origami 140 or, more generally, positive detection supramolecular structures 248 herein, are released (step 132) and subsequently isolated (step 136), aspects of which are illustrated in FIGS. 6 and 7.

By way of example, and turning to FIG. 6, the topmost illustration illustrates both localization supramolecular structures 176 and positive detection supramolecular structures 248 (i.e., detection supramolecular structures which have bound to a localized analyte of interest 190A) concurrently bound to the analyte of interest 190A, such as at different regions or domains. The bottom illustration of FIG. 6 illustrates a step corresponding to step 132 of FIG. 1 in which the positive detection supramolecular structures 248 are released from the analyte of interest 190A via activation or operation of the detachment site 280 present in the linkage to the affinity binder 230. As noted herein, detachment of the affinity binder 230 from the core structure 220 of the positive detection supramolecular structure 248 may be accomplished via any suitable operation including, but not limited to, photo-cleavage, chemical cleavage, or strand displacement.

As shown in FIG. 6, after displacement and release of the positive detection supramolecular structures 248, the positive detection supramolecular structures 248 are unlinked from the analyte of interest 190A, which remains bound to the hydrogel matrix 160, and may be subsequently washed or flowed through the hydrogel matrix 160 and isolated for subsequent processing. By way of example, the unbound positive detection supramolecular structures 248 may be flowed through the hydrogel matrix 160 within the flow channel 164 to a collection container or vessel for use in an assay step. In the context of a microfluidic chip or device, the positive detection supramolecular structures 248 may be flowed to a fluidically connected chamber having a detection substrate as discussed herein. This aspect is illustrated in FIG. 7, in which the topmost illustration depicts the release step 132 (FIG. 1) by which the positive detection supramolecular structures 248 are released from being bound to the molecules of the analyte of interest 190A (which remain bound to the localization supramolecular structures 176 and the hydrogel matrix 160) and the bottommost illustration depicts the released positive detection supramolecular structures 248, with no bound analyte of interest 190A or affinity binders 230, being separated from the hydrogel matrix 160 for isolation. At this stage, the flow channel 164, hydrogel matrix 160, localization supramolecular structures 176, and analyte of interest 190A can be discarded, as illustrated in FIG. 7.

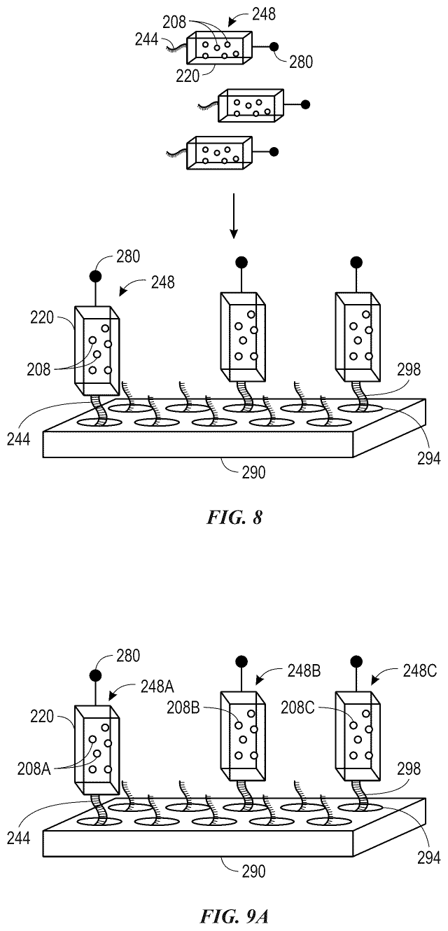

Turning back to the DNA origami-based protein assay example of FIG. 1, the isolated positive detection origami 140 of the depicted example may be flowed over or otherwise applied to a detection substrate to which the positive detection origami 140 are configured to bind or otherwise attach. This is illustrated in the example of FIG. 1 at step 144 at which the positive detection origami 140 are hybridized with the detection substrate, such as to attachment sites, wells, or pads of the detection substrate. This is further illustrated in FIG. 8, in which in the top illustration the unbound positive detection supramolecular structures 248 are illustrated, such as in a solution phase prior to exposure to a detection substrate 290. In the bottom illustration of FIG. 8, the positive detection supramolecular structures 248 are shown after exposure to the detection substrate 290, which in the depicted example includes a plurality of attachment sites 294 each having a substrate attachment molecule 298 configured to interact with and chemically bind to the anchor molecules 244 of the positive detection supramolecular structures 248. In the depicted example the substrate attachment molecule 298 is depicted as a single strand nucleic acid molecule of a specific sequence that is complementary, at least in part, to a single strand nucleic acid molecule forming the anchor molecule or barcode 244 of the respective positive detection supramolecular structures 248.

Turning back to the particular example of FIG. 1, once hybridized to the detection substrate 290, the positive detection origami 140 may be counted (step 148) to determine the presence and/or quantity of the analyte molecules of interest 190A in the original sample 100. As noted herein, due to the association of a single affinity binder 230 to each detection supramolecular substrate 200, a 1:1 ratio between detection supramolecular substrate 200 and molecules of analyte of interest 190A is obtained such that counting the positive detection origami 140 is functionally equivalent to counting the individual molecules of analyte of interest 190A in the sample 100. Therefore, in this example, the result of the count may constitute an assay result 152 corresponding to the presence and/or quantity (absolute or relative) of the analyte of interest within the sample 100. Of note, at the hybridization and count steps, none of the analyte of interest 190A is present on the positive detection supramolecular structures 248 or otherwise bound to the detection substrate 290. Indeed, the analyte of interest 190A, which remains bound to the localization supramolecular structures 176 and hydrogel matrix 160, may be discarded prior to steps 144 and 168 associated with the assay. It may be noted that the absence of binding of the analyte of interest 190A, or more generally the absence of the analyte of interest 190A in the presence of the detection substrate 290 allows for a cleaner substrate with no specific binding or interaction with the analyte(s). This in turn may allow the detection substrate to be renewed and regenerated for use in subsequent assay operations.

In practice, detecting and/or counting the positive detection supramolecular structures 248 may be accomplished via the excitation and emissions of the fluorescent molecules 208 present on the detection supramolecular structures 100. Thus, the type of emission (e.g., wavelength or frequency), the intensity of emission, and so forth, may be used to detect and/or count positive detection supramolecular structures 248, each corresponding to an analyte of interest 190A. In embodiments where each positive detection supramolecular structures 248 was bound to a single analyte of interest molecule 190A and where a single positive detection supramolecular structures 248 binds to each attachment site 294, a count of emitting attachment sites can provide a count of the number of molecules of the analyte of interest present in the sample 100.

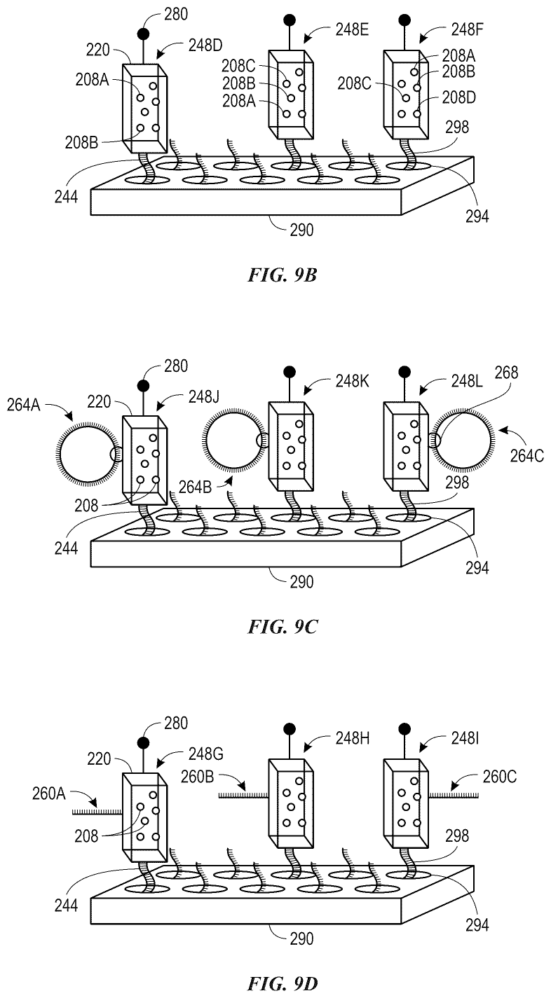

Turning to FIGS. 9A through 9D, in certain embodiments hybridization and counting steps may be multiplexed so as to allow multiple analytes of interest 190A to be assayed in a given operation. By way of example, in processing a given sample 100, different sets of localization supramolecular structures 176 and detection supramolecular structures 200 may be employed wherein each set has respective affinity binders 184, 230 for different analytes 190. In such embodiments, the respective positive detection supramolecular structures 248 may be distinguished during the assay operation based upon different fluorophores 208 or by different ratios or combinations of fluorophores 208. By way of example, as shown in FIG. 9A, positive detection supramolecular structures 248A, 248B, and 248C may have respective binding specificities to different analytes of interest. Correspondingly, the positive detection supramolecular structures 248A 248B, and 248C may be characterized by different attached fluorophores 208A, 208B, and 208C respectively which differ in their excitation wavelength response and/or emission wavelengths, thereby allowing the respective positive detection supramolecular structures 248 to be differentiated at the detection substrate 290.

While FIG. 9A depicts an embodiment in which each positive detection supramolecular structures 248 is associated with a single fluorophore type (e.g., red, blue, green, etc.), FIG. 9B instead depicts an embodiment in which the respective positive detection supramolecular structures 248D, 248E, and 248F, each of which may be characteristic of (i.e., have had a specific binding affinity for) a different analyte of interest, are characterized by a different combination or ratio of fluorophores 208. In this manner, by combining fluorophores 208 on the positive detection supramolecular structures 248, a greater number of analytes of interest 190A may be assayed at one time as even a limited number of fluorophore types can give rise to a large number of fluorophore combinations (i.e., permutations).

In a further embodiment, FIG. 9C depicts an implementation in which the respective positive detection supramolecular structures 248J, 248K, and 248L, each of which may be characteristic of (i.e., have had a specific binding affinity for) a different analyte of interest, are characterized by a different attached barcode or unique sequences (e.g., nucleic acid sequences) that may be incorporated onto a circular template 264A, 264B, and 264C suitable for rolling circle amplification. By incubating the circular templates 264 in solution with suitable transcription precursors and enzymes, a continuous strand of sequential barcode sequences is generated so as to form a nanoball connected to the respective positive detection supramolecular structures 248. In this manner, the number of barcode sequences attached to the positive detection supramolecular structures can be controlled by controlling the incubation environment (e.g., temperature, precursor concentrations or amounts, and so forth) and/or the duration of the incubation.

In this example, a rolling circle amplification (RCA) template tag or polymer linker 268 is linked to the supramolecular structure (such as at a particular or targeted staple strand), such as via complementary sequence pairing or other suitable chemical bonding. The RCA template 264 (e.g., circular template) itself may then be linked to the tag 268 via complementary sequence pairing or other suitable chemical bonding. The RCA template linker 268 and the affinity binder linker may be separated by selective placement on opposing or different surfaces of the supramolecular structure (e.g., a DNA origami), such as by placement on a top surface and a bottom surface or a top surface and a side surface respectively.

In accordance with this embodiment, an example of an RCA template 264 comprises unique identifiers or barcodes (e.g., nucleic acid sequences) incorporated in the circular template 264 in a continuous or linked sequential arrangement. The RCA template 264 may consist of single or multiple copies of each barcode sequence. By way of example, by having different numbers of copies of each barcode sequence in each circular template 264, a respective ratio of barcode signals may be generated using a given circular template, with the ratio conveying or characterizing the analyte molecule affinity for a given detection supramolecular structure 200 to which the circular template is attached.

In one example, the circular template may comprise a circularized single-stranded DNA (i.e., ssDNA) strand having a primer region followed by a known or designed number of unique barcode region sequences. Each barcode region sequence may be separated a spacer sequence or region. Amplification of the RCA template 264 results in formation of an amplicon comprising the transcribed barcode sequences in the order and number in which they are present on the RCA template 264 and, in one embodiment, causing the formation of a nanoball. That is, the nanoball(s) is formed as an amplicon of the RCA template and may be formed as a continuous strand comprising a repeated and sequential sequence of the plurality of copies of the two or more barcode sequences. In one embodiment a respective and corresponding fluorophore is bound by complementary pairing to each copy of a respective sequence of the nanoball as part of a detection step. In one non-limiting example, the nanoball 156 has a hydrodynamic radius of between approximately 100 nm to approximately 2 μm.

Association of respective complementary fluorophores (e.g., before, during, or after exposure to the analyte containing sample) causes a respective fluorescent molecule to be associated with each transcribed barcode sequence present in the nanoball. The duration of the incubation period over which the amplification is allowed to occur (as well as other reaction controlling characteristics), therefore, can directly determine the number of copies made of the RCA template 264, and therefore the resulting optical signal that may be associated with the nanoball. In this manner, measurable signal per analyte molecule of interest 190A may be increased or decreased based on the parameters of the detection mechanism so as to optimize the classification and/or quantification operation with respect to the analyte molecule. By way of example, in instances where the analyte molecule of interest is at very low levels or concentrations, down to single-molecule quantification, a high-signal level per positive detection supramolecular structures 248 (and correspondingly per analyte molecule) may be useful. Conversely, in instances where the analyte-molecule is at relatively higher levels or concentrations, a lower-signal level per positive detection supramolecular structures 248 may be sufficient, or even desirable. In this manner, the detectable signal per positive detection supramolecular structures 248 can be customized or optimized as indicated by the use case or sample context. As noted above, this may provide a degree of signal optimization.

By way of further discussion, as noted herein the relative proportions of different barcode sequences may be expressed in such an amplicon (such as a nanoball) and may be read out and the results used to identify the analyte specificity of a respective positive detection supramolecular structures 248. This may allow a limited number of barcode sequences to, in various combinations, be used to identify a large number of analyte specificities. By way of example, a ratio of two barcode sequences A and B of 1:1 may correspond to a respective analyte specificity of a positive detection supramolecular structures 248. However, the relative proportion of each barcode sequence may be varied (e.g., 2:1, 3:1, 4:1, 1:2, 1:3, 1:4, and so forth) so as to create various unique measurable ratios, each corresponding to different analyte specificities while using only two barcode sequences. By increasing the number of barcode sequences employed, an even greater number of unique barcode sequence ratios may be achieved. For example, for barcode sequences A, B, and C, example ratios may include, but are not limited to: 1:1:1, 1:1:2, 1:2:1, 2:1:1, 1:1:3, 1:3:1, 3:1:1, 1:2:3, 2:1:3, 1:3:2, 2:3:1, 3:1:2, 3:2:1, and so forth. The number of permutations of ratios may be increased by adding additional numbers of available barcode sequences (e.g., D and E) such that the number of ratio permutations allows for a large number of unique ratios of barcode sequences each uniquely identifying an analyte specificity of respective detection supramolecular structures 100. Statistically, this may be represented as:

#permutations=(n−2)³ (1)

where the number of permutations corresponds to the number of possible unique nanoballs based upon ratios of barcodes and n is the number of different barcode sections or sequences.

The strand from which the nanoball is formed comprises multiple, linearly sequential copies of the barcode sequences as specified by the RCA template 264. In this example, the barcode sequences encoded by the nanoball 156 are complementary to respective fluorophores 208 that comprise both a polymer strand complementary to respective barcode sequences as well as an attached fluorescent molecule characteristic of a respective set of fluorophores 208. Based on their complementary relationship a respective fluorophore 208 may bind to the complementary barcode sequence to form a respective fluorescent conjugate on the nanoball, and thereby on the positive detection supramolecular structures 248. In practice, the fluorophores 208 may be bound to the barcode sequences before, during or after binding of analyte molecules to the detection supramolecular structures 100.

Turning to FIG. 9D, in a further alternative, the respective positive detection supramolecular structures 248 may be distinguished during the assay binder based upon different decode tags or sequences 260 (e.g., barcode sequences), each comprising a single or multiple strands having unique decode sequences characteristic of the affinity binder 230 with which each positive detection supramolecular structures 248 was previously associated. By way of example, as shown in FIG. 9D, positive detection supramolecular structures 248G, 248H, and 248I may have respective binding specificities to different analytes of interest. Correspondingly, the positive detection supramolecular structures 248G, 248H, and 248I may be characterized by different decode strands 260A, 260B, and 260C respectively differing in the respective set or series of subsequences (e.g., barcode sequences) on the strands.

As in the preceding example, the barcode sequences encoded on the strands 260 are complementary to respective fluorophores 208 that comprise both a polymer strand complementary to respective barcode sequences as well as an attached fluorescent molecule characteristic of a respective set of fluorophores 208. Based on their complementary relationship a respective fluorophore 208 may bind to the complementary barcode sequence to form a respective fluorescent conjugate on the decode strand 260, and thereby on the positive detection supramolecular structures 248. In practice, the fluorophores 208 may be bound to the barcode sequences before, during or after binding of analyte molecules to the detection supramolecular structures 100.

It may be noted that certain of the preceding multiplex examples are primarily beneficial in a context in which the attachment of the detection supramolecular structures 248 to the detection substrate 290 is random, and therefore the decode information must be obtained from the positive detection supramolecular structures 248 themselves. Conversely, when attachment of the detection supramolecular structures 248 to the attachment sites 294 of the detection substrate 290 is not random, such as when different attachment sites 294 are configured (e.g., based upon their respective substrate attachment molecules 298) to bind selectively to particular positive detection supramolecular structures 248 based on the analyte specificity of the supramolecular structures, decode may be performed based upon which attachments sites 294 exhibit fluorescence. That is, the detection substrate 290 is “mapped” so that different attachment sites 294 correspond to different analytes of interest 190A, and differentiation of different types of positive detection supramolecular structures 248 is not necessary.

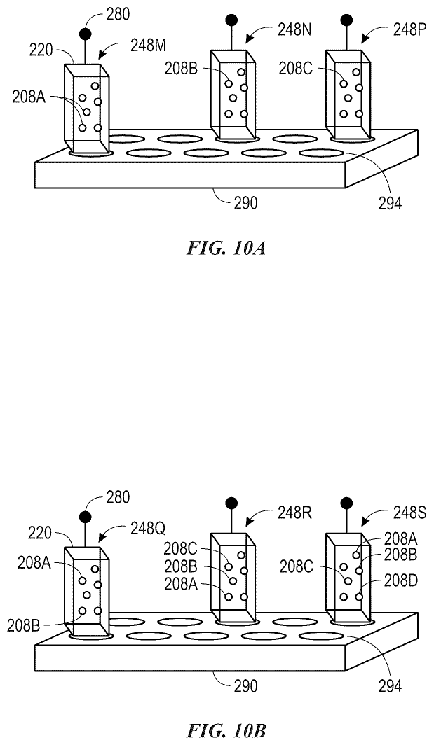

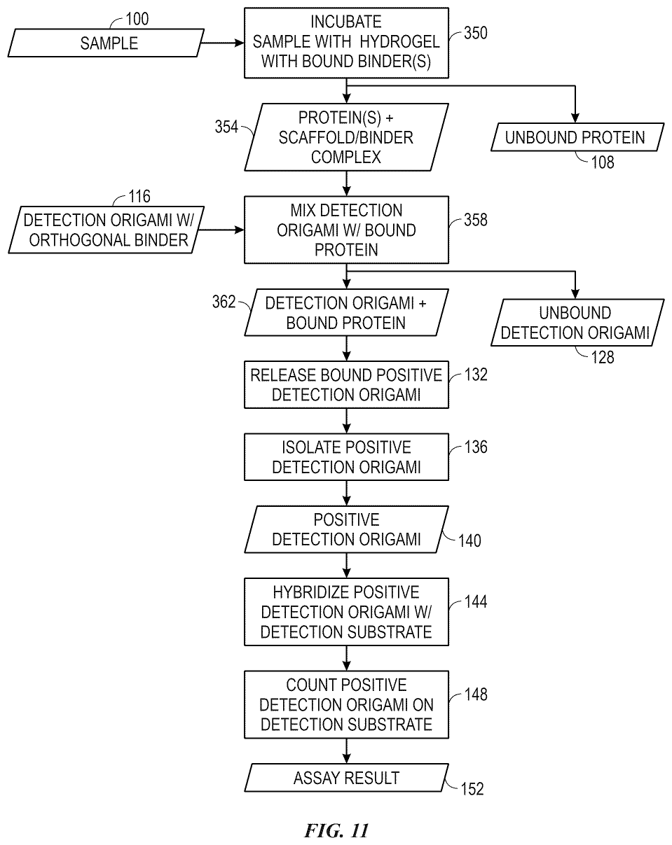

With this in mind, and turning to FIGS. 10A and 10B, two further examples are provided of embodiments in which hybridization and counting steps may be multiplexed so as to allow multiple analytes of interest 190A to be assayed in a given operation. By way of example, in the implementations depicted in FIGS. 10A and 10B the positive detection supramolecular structures 248 (e.g., reporter DNA origami boxes or reporter molecules) associate with the detection substrate 290 without the positive detection supramolecular structures 248 being attached to the attachment sites 294 via complementary nucleic acid (e.g., DNA) oligomers. Instead, the supramolecular structures 248 non-specifically interact with (e.g., bind to) the attachment sites 294 based on the presence of bridging salt molecules, such as ammonium persulfate (APS).

As may be appreciated, the non-specific nature of the interaction of the supramolecular structures 248 with the attachment sites 294 offers certain advantages. For example, because a given species of positive detection supramolecular structure 248 does not have to search for a corresponding and specific attachment site 294, the respective supramolecular structures can more quickly associate with an available attachment site 294, thereby reducing the incubation time required to get a measurable number of supramolecular structures 248 to associate with attachment sites 294. In practice, this may allow operation in incubation regimes significantly shorter than what would otherwise be required to allow for complementary (or other specific) binding interactions.

In these examples, the positive detection supramolecular structures 248 are incubated with the detection substrate 290 as in preceding examples. The attachment (e.g., binding) sites 294 on the detection substrate (e.g., chip substrate) are configured (based on size and/or geometry) so as to have physical dimensions or shapes that limit binding opportunities of the positive detection supramolecular structures 248 such that multiple supramolecular structures 248 cannot, or are discouraged from, attaching to (e.g., “land on”) a respective attachment site 294 simultaneously. Furthermore, simultaneous landing events, if they occur, can be discriminated based on the recorded fluorescence intensity per site, as discussed below.

Turning to FIG. 10A, in this example the identity of the analyte associated with respective positive detection supramolecular structures 248M, 248N, and 248P may be inferred based on the fluorescent color associated with the respective positive detection supramolecular structure 248, and the total number of such supramolecular structures 248 counted on the surface of the detection substrate 290 is directly proportional to the concentration of the analyte assayed.

Similarly, and as illustrated in FIG. 10B, the positive detection supramolecular structures 248 also associate with the respective attachment sites 294 without the assistance of complementary nucleic acid oligomers. Unlike the example illustrated in FIG. 10A, in the example of FIG. 10B the identity of individual analytes is encoded by a different combination or ratio of fluorophores 208 (e.g., fluorophores 208A, 208B, 208C, 208D) associated with the respective positive detection supramolecular structures 248Q, 248R, and 248S.

In contrast to the implementations described with respect to FIGS. 9A-9D, the embodiments illustrated with respect to FIGS. 10A and 10B obviate the need for pre-processing of the attachment site 294 with complementary oligonucleotides. As will be appreciated, however, there is a corresponding lack of specificity in the attachment of the positive detection supramolecular structures 248 to the attachment sites 294. However, in a low-plex regime, one need only discriminate for single landing events (as opposed to a multiplicity of boxes landing on the same pad) and determination of the identity of a respective analyte is a direct function of the fluorescent channel in which a respective positive detection supramolecular structures 248 is detected. Further, the number of positive detection supramolecular structures 248 binding to a given attachment site 294 may be determined based on the fluorescent intensity observed at the attachment site 294 in question. As will be appreciated from the discussion herein, this result is possible in the case of nucleic acid origami based supramolecular structures 248 because each such structure is loaded with n fluorophores 208, with n being a known or specified engineering parameter, such as 56 fluorophores, though more or less fluorophores may instead be specified. Because of the relatively large number of fluorescent dye molecules per supramolecular structure 248 and that the observed fluorescent intensity is quantized (i.e., one can have an integer number of supramolecular structures 248, but not a fractional number of supramolecular structures 248), it is possible to quantify the number of positive detection supramolecular structures 248 per attachment site 294 and to sum up all of the respective types (i.e., species) of positive detection supramolecular structures 248 (each corresponding to a different analyte) across the detection substrate 290. Correspondingly, such approaches may have utility and benefit in low-plex assay contexts by providing a cost-effective approach to detection.

As may be appreciated, in other implementations the above-described approach may be varied. By way of example, the above-described approach utilizes localization supramolecular structures 176 that help address potential steric hinderance issues that might arise from some portion of the affinity binders used to localize molecules of the analyte of interest being too tightly spaced on the hydrogel matrix 160, but at the cost of increased complexity. Alternatively, as described in the following example implementation, if such steric hinderance is not a factor and/or the increased complexity associated with the incorporation of localization supramolecular structures 176 is undesired, other approaches may be employed. By way of example, and turning to FIG. 11 and the associated figures, in a further embodiment the affinity binders 184 used to localize the molecules of the analyte of interest 190A to the hydrogel matrix 160 may be attached to the hydrogel matrix 160 and not to an intervening intermediary structure, such as the localization supramolecular structure 176 described in the preceding example.

With this background and context in mind, and turning to the process flow of FIG. 11, in the depicted process flow of one implementation, a sample 100 containing one or more analytes of interest, is incubated (step 350) with a scaffold structure(s) (e.g., a hydrogel matrix 160) to which localization affinity binders 184 are linked or otherwise attached, which may jointly be referred to, in one such embodiment, as a scaffold-binder complex.

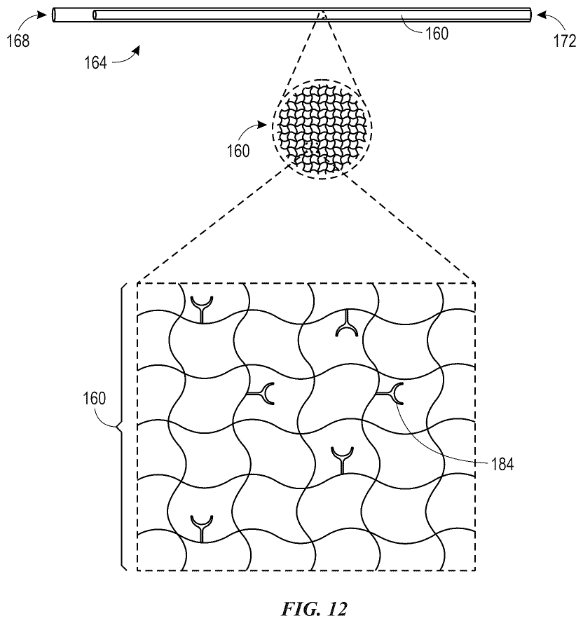

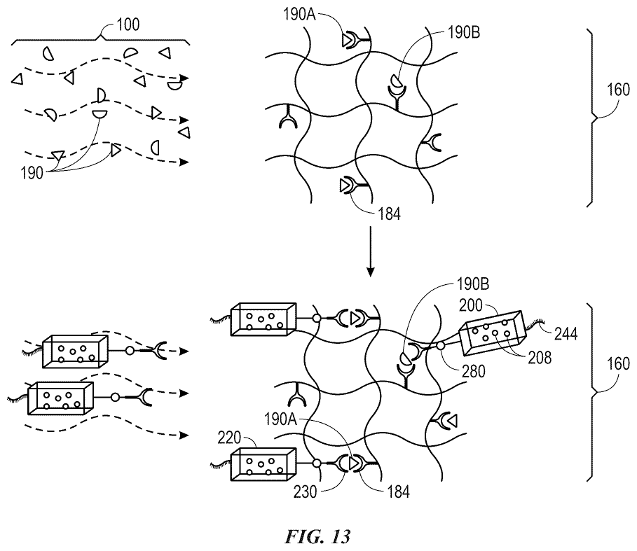

With this in mind, and turning to FIG. 12 and the topmost illustration of FIG. 13, possible implementations are illustrated to better illustrate aspects of this step. Turning to FIG. 12, a hydrogel matrix 160 is illustrated as being disposed within a flow channel 164 (e.g., a capillary tube or channel within a microfluidic device or chip) having an input port 168 and an output port 172 through which the sample may be flowed. By way of example a hydrogel, as used herein, may be a polyacrylamide, polyimide, or other suitable matrix).

As shown in the successive expanded views of FIG. 12, the hydrogel matrix 160 may be complexed with localization affinity binders 184. As described herein, each localization affinity binder 184 (or reactive group) is specific to the analyte molecule of interest 190A, such as to a specific binding region or domain on the analyte molecule of interest 190A. As discussed herein, a respective analyte molecule may bind with specificity to the affinity binder 184 or reactive group through a chemical bond. In some embodiments, the affinity binder 184 or reactive group comprises a protein, a peptide, an antibody, an aptamer (e.g., RNA and DNA-based aptamers), a darpin, a polymer like PEG, or combinations thereof.

The hydrogel matrix 160 may be complexed with localization affinity binders 184 via a polymer strand or linker molecule. For example, in certain embodiments attachment of the localization affinity binder 184 is via a linker structure or strand that chemically links to random or targeted (i.e., non-random) locations (e.g., complementary linker structures or strands) provided on the hydrogel matrix 160. By way of example, and in the context of affinity binders 184 (e.g., antibodies) linked to the hydrogel matrix 160, such linkage may be accomplished using complementary nucleic acid strand pairing or other complementary pairing, via covalent or other chemical bond formation, and/or by any other suitable attachment mechanism. In such embodiments, the localization affinity binder 184 may be bound or attached to respective binding sites of the hydrogel matrix. Such surfaces or substrates may comprise a plurality of binding sites, wherein each binding site is configured to link with a corresponding localization affinity binder 184. Conversely, in other embodiments the localization affinity binders 184 may attached to the hydrogel matrix 160 at random locations or sites (i.e., in an untargeted or uncontrolled manner). When bound to a hydrogel matrix 160, the localization affinity binders 184, and any analyte bound to the localization affinity binders 184, may be substantially fixed in location with respect to the hydrogel matrix 160. By way of example, each localization affinity binder 184 may include or incorporate a single strand nucleic acid attachment strand that is complementary to a complement strand of nucleic acid seeded on the hydrogel matrix 160. The resulting hydrogel matrix 160/localization affinity binder 184 complex has binding specificity to a specific analyte molecule, as determined by the affinity binder 184, and in the presence of the specific analyte molecule will bind to such molecules.

In the topmost illustration of FIG. 13, it may be appreciated that the localization binders 184, when binding an analyte molecule of interest 190A, thereby localizes the bound analyte of interest 190A to the substrate (here the hydrogel matrix 160) for subsequent operations, as discussed herein. As shown in FIG. 13, which illustrates aspects of the step 350, a sample 100 comprising analytes 190, including analytes of interest 190A, is exposed to the hydrogel matrix 160 in this embodiment. All or some portion of the analyte molecules of interest 190A bind to the affinity binders 184 present on the hydrogel matrix 160. By way of example, the analyte molecules of interest 190A may bind via a specific interaction to the affinity binders 184 due to the specificity of the affinity binder 184 for the analyte of interest 190A. Some portion of other analyte molecules (i.e., analytes not of interest 190B) may also interact with the affinity binders 184 via non-specific interactions, but such interactions, if present will be weak and limited in number.

After an incubation period, the unbound analyte molecules may be flowed or washed away through the flow channel 164 so as to remove unbound analyte molecules, such as analytes not of interest 190B. At this stage, therefore, primarily analytes of interest 190A remain bound to the substrate (e.g., hydrogel matrix 160), with unbound analytes having been removed via the output port 172 of the flow channel 164.

Turning back to FIG. 11, this is illustrated in the example, process flow in the context of a protein assay. Hence unbound protein 108 is shown as being separated and removed subsequence to the incubation step 350, leaving the protein of interest bound to the scaffold-binder complex (complex 354 in the aggregate).

As illustrated in FIG. 11, at a next stage a detection supramolecular structures 200, in the form of detection origami 116 in the depicted example, are flowed through the flow channel 164 and hydrogel matrix 160 so as to mix (step 358) with the protein scaffold/binder complex 354 formed by the localization affinity binders 184 attached to the hydrogel matrix 160 and bound analyte 190A as shown in FIG. 13. In the depicted example, the localization affinity binders 184 and 230 present on the hydrogel matrix 160 and the detection supramolecular structures 200 respectively are specific to the analyte of interest 190A at different regions or domains (i.e., orthogonal binders). In this manner, both the hydrogel matrix 160 and the detection supramolecular structure 200 can simultaneously bind to a respective analyte of interest 190A.

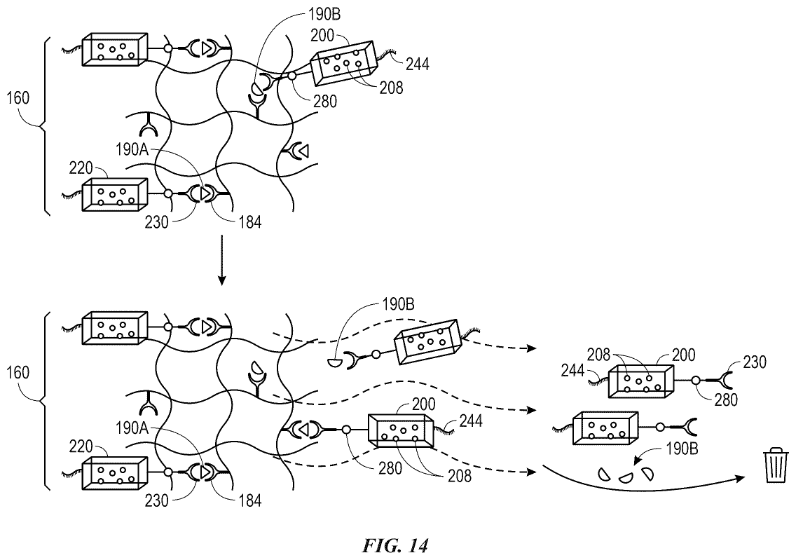

This is further illustrated in FIG. 13, in which the topmost illustration illustrates the protein scaffold/binder complex formed by the analyte of interest 190 and the localization affinity binders 184 attached to the hydrogel matrix 160 prior to addition of the detection supramolecular structures 200 (top) and after addition of the detection supramolecular structures 200 (bottom). As shown in this illustration, analytes of interest 190A bound to the localization affinity binders 184 at this stage are also bound at different regions or domains to the detection supramolecular structures 200, such as via a different and distinct specific interaction. As shown in FIG. 14 (bottom) unbound detection supramolecular structures 200 may be washed away (e.g., flowed through the flow channel 164). Turning back to FIG. 11, in the detector origami implementation depicted, these outcomes are illustrated respectively by the unbound detection origami 128 that are washed away and the remaining detection origami/bound protein complex 362 that remain for further processing.

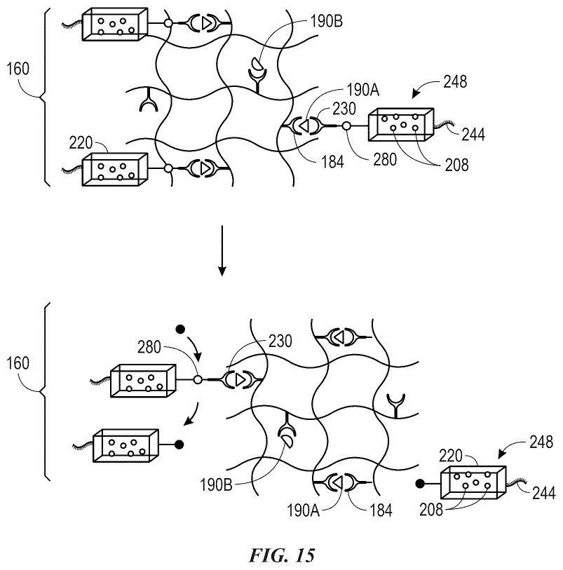

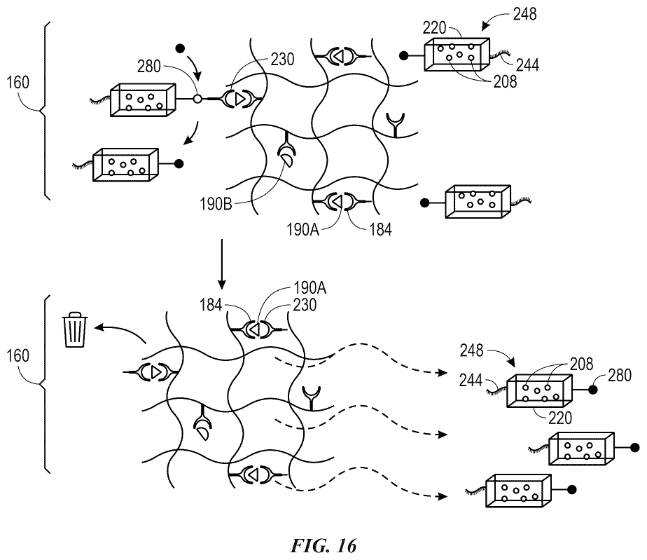

As further shown in FIG. 11, in a next step the detection origami that were successfully bound to analyte of interest at step 358, referred to as positive detection origami 140 or positive detection supramolecular structures 248 herein, are released (step 132) and subsequently isolated (step 136), aspects of which are illustrated in FIGS. 15 and 16.

By way of example, and turning to FIG. 15, the topmost illustration illustrates both localization affinity binders 184 and positive detection supramolecular structures 248 (i.e., detection supramolecular structures which have bound to a localized analyte of interest 190A) concurrently bound to the analyte of interest 190A, such as at different regions or domains. The bottom illustration of FIG. 15 illustrates a step corresponding to step 132 of FIG. 11 in which the positive detection supramolecular structures 248 are released from the analyte of interest 190A via activation or operation of the detachment site 280 present in the linkage to the affinity binder 230. As noted herein, detachment of the affinity binder 230 from the core structure 220 of the positive detection supramolecular structure 248 may be accomplished via any suitable operation including, but not limited to, photo-cleavage, chemical cleavage, or strand displacement.

As shown in FIG. 15, after displacement and release of the positive detection supramolecular structures 248, the positive detection supramolecular structures 248 are unlinked from the analyte of interest 190A, which remains bound to the hydrogel matrix 160, and may subsequently be washed or flowed through the hydrogel matrix 160 and isolated for subsequent processing. By way of example, the unbound positive detection supramolecular structures 248 may be flowed through the hydrogel matrix 160 within the flow channel 164 to a collection container or vessel for use in an assay step. In the context of a microfluidic chip or device, the positive detection supramolecular structures 248 may be flowed to a fluidically connected chamber having a detection substrate as discussed herein. This aspect is illustrated in FIG. 16, in which the topmost illustration depicts the release step 132 (FIG. 11) by which the positive detection supramolecular structures 248 are released from being bound to the molecules of the analyte of interest 190A (which remain bound to the localization affinity binders 184 and the hydrogel matrix 160) and the bottommost illustration depicts the released positive detection supramolecular structures 248, with no bound analyte of interest 190A or affinity binders 230, being separated from the hydrogel matrix 160 for isolation. At this stage, the flow channel 164, hydrogel matrix 160, localization affinity binders 184, and analyte of interest 190A can be discarded, as illustrated in FIG. 16.

Turning back to the DNA origami-based protein assay example of FIG. 11, and as previously described, the isolated positive detection origami 140 of the depicted example may be flowed over or otherwise applied to a detection substrate 290 to which the positive detection origami 140 are configured to bind or otherwise attach. This is illustrated in the example of FIG. 11 at step 144 at which the positive detection origami 140 are hybridized with the detection substrate 290, such as to attachment sites 294, wells, or pads of the detection substrate 290.

As shown in the particular example of FIG. 11, once hybridized to the detection substrate 290, the positive detection origami 140 may be counted (step 148) to determine the presence and/or quantity of the analyte molecules of interest 190A in the original sample 100. As noted herein, due to the association of a single affinity binder 230 to each detection supramolecular substrate 200, a 1:1 ratio between detection supramolecular substrate 200 and molecules of analyte of interest 190A may be obtained such that counting the positive detection origami 140 is functionally equivalent to counting the individual molecules of analyte of interest 190A in the sample 100. Therefore, in this example, the result of the count may constitute an assay result 152 corresponding to the presence and/or quantity (absolute or relative) of the analyte of interest within the sample 100. Of note, at the hybridization and count steps, none of the analyte of interest 190A is present on the positive detection supramolecular structures 248 or otherwise bound to the detection substrate 290. Indeed, the analyte of interest 190A, which remains bound to the localization supramolecular structures 176 and hydrogel matrix 160, may be discarded prior to steps 144 and 168 associated with the assay. It may be noted that the absence of binding of the analyte of interest 190A, or more generally the absence of the analyte of interest 190A in the presence of the detection substrate 290 allows for a cleaner substrate with no specific binding or interaction with the analyte(s). This in turn may allow the detection substrate to be renewed and regenerated for use in subsequent assay operations.

As previously described, in practice the steps of detecting and/or counting the positive detection supramolecular structures 248 may be accomplished via the excitation and emissions of the fluorescent molecules 208 present on the detection supramolecular structures 100. Thus, the type of emission (e.g., wavelength or frequency), the intensity of emission, and so forth, may be used to detect and/or count positive detection supramolecular structures 248, each corresponding to an analyte of interest 190A. In embodiments where each positive detection supramolecular structures 248 was bound to a single analyte of interest molecule 190A and where a single positive detection supramolecular structures 248 binds to each attachment site 294, a count of emitting attachment sites can provide a count of the number of molecules of the analyte of interest present in the sample 100. Further, as described herein, in certain embodiments hybridization and counting steps may be multiplexed so as to allow multiple analytes of interest 190A to be assayed in a given operation.

While the preceding relate various examples in the context of a flow channel, such as a microfluidic flow channel or capillary tube, in other contexts localization of the analyte of interest may be performed with respect to other substrates. By way of example, beads, such as magnetic beads, may be used as a substrate for binding and localization of the analyte of interest 190A. In practice, such beads may be coated with streptavidin and/or may otherwise be conducive to attaching monoclonal antibodies or nucleic acid strands (e.g., DNA). Such approaches may provide flexibility relative to other approaches in terms of not being limited to a particular microfluidic structure or context.

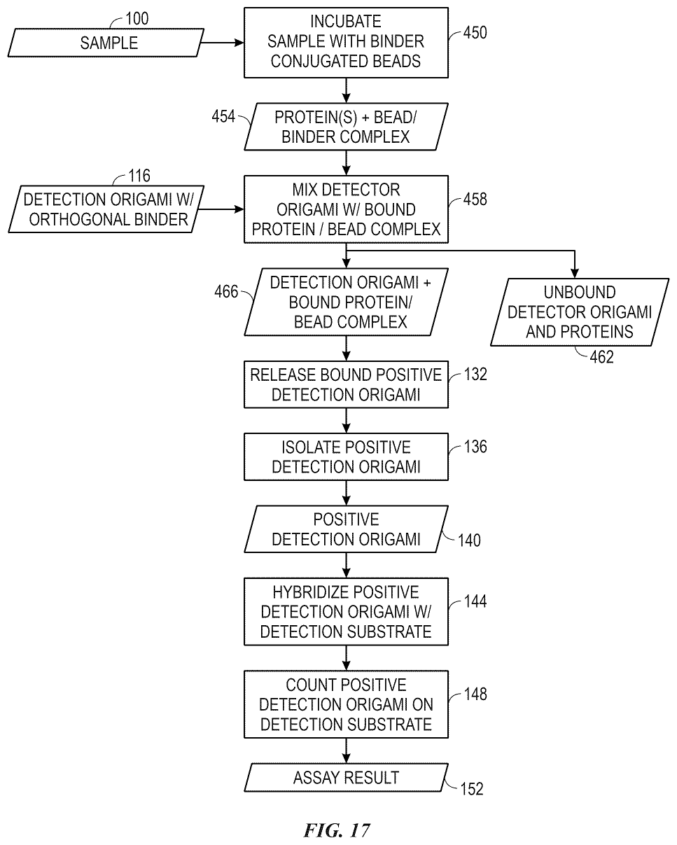

By way of example, and turning to FIG. 17 and the associated figures, in a further embodiment the affinity binders 184 used to localize the molecules of the analyte of interest 190A may be attached or otherwise linked to beads, such as magnetic beads. As shown in the process flow of FIG. 17, in one implementation, a sample 100 containing one or more analytes 190, is incubated (step 450) with binder (e.g., affinity binder) conjugated beads (e.g., magnetic or other coated beads, such as streptavidin coated beads). By way of example, the beads may be conjugated with or otherwise attached to antibodies or other affinity binders 184, such as via known techniques, to form a bead-binder complex.

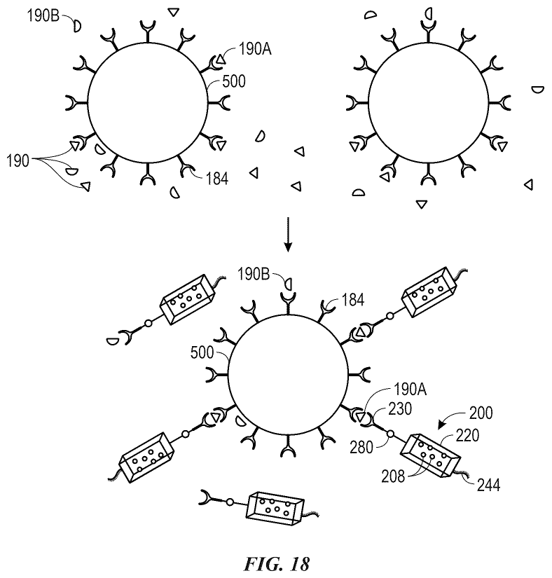

With this in mind, and turning to FIG. 18, possible implementations are illustrated to better illustrate aspects of this step. Turning to FIG. 18, beads 500 are illustrated as being in solution with a sample 100 having analytes 190, including analytes of interest 190A and analytes that are not of interest 190B. As shown in this example each bead 500 may be complexed with localization affinity binders 184. As described herein, each localization affinity binder 184 (or reactive group) is specific to an analyte molecule of interest 190A, such as to a specific binding region or domain on the analyte molecule of interest 190A. As discussed herein, a respective analyte molecule may bind with specificity to the affinity binder 184 or reactive group through a chemical bond. In some embodiments, the affinity binder 184 or reactive group comprises a protein, a peptide, an antibody, an aptamer (e.g., RNA and DNA-based aptamers), a darpin, a polymer like PEG, or combinations thereof.

The beads 500 may be complexed with localization affinity binders 184 via a polymer strand or linker molecule. For example, in certain embodiments attachment of the localization affinity binder 184 is via a linker structure or strand that chemically links to random or targeted (i.e., non-random) locations (e.g., complementary linker structures or strands) provided on the bead 500. By way of example, and in the context of affinity binders 184 (e.g., antibodies) linked to the beads 500, such linkage may be accomplished using complementary nucleic acid strand pairing or other complementary pairing, via covalent or other chemical bond formation, and/or by any other suitable attachment mechanism. In such embodiments, the localization affinity binder 184 may be bound or attached to respective binding sites on the beads 500. Such surfaces or substrates may comprise a plurality of binding sites, wherein each binding site is configured to link with a corresponding localization affinity binder 184. Conversely, in other embodiments the localization affinity binders 184 may attached to the beads 500 at random locations or sites (i.e., in an untargeted or uncontrolled manner). When bound to a bead 500, the localization affinity binders 184, and any analyte bound to the localization affinity binders 184, may be substantially fixed in location with respect to the bead 500. By way of example, each localization affinity binder 184 may include or incorporate a single strand nucleic acid attachment strand that is complementary to a complement strand of nucleic acid seeded on the bead 500. The resulting bead 500/localization affinity binder 184 complex has binding specificity to a specific analyte molecule, as determined by the affinity binder 184, and in the presence of the specific analyte molecule will bind to such molecules.

In the topmost illustration of FIG. 18, it may be appreciated that the localization binders 184, when binding an analyte molecule of interest 190A, thereby localizes the bound analyte of interest 190A to the substrate (here a bead 500) for subsequent operations, as discussed herein. As shown in FIG. 18, which illustrates aspects of the step 350, a sample 100 comprising analytes 190, including analytes of interest 190A, is exposed to the beads 500 in this embodiment. All or some portion of the analyte molecules of interest 190A bind to the affinity binders 184 present on the beads 500. By way of example, the analyte molecules of interest 190A may bind via a specific interaction to the affinity binders 184 due to the specificity of the affinity binder 184 for the analyte of interest 190A to form a protein+bead/binder complex 454. Some portion of other analyte molecules (i.e., analytes not of interest 190B) may also interact with the affinity binders 184 via non-specific interactions, but such interactions, if present will be weak and limited in number.

As illustrated in FIG. 17, at a next stage a detection supramolecular structures 200, in the form of detection origami 116 in the depicted example, are added to the solution so as to mix (step 458) with the protein+bead/binder complexes 454 formed by the localization affinity binders 184 attached to the beads 500 and bound analyte 190A as shown in FIG. 18. In the depicted example, the localization affinity binders 184 and 230 present on the beads 500 and the detection supramolecular structures 200 respectively are specific to the analyte of interest 190A at different regions or domains (i.e., orthogonal binders). In this manner, both the beads 500 and the detection supramolecular structure 200 can simultaneously bind to a respective analyte of interest 190A.

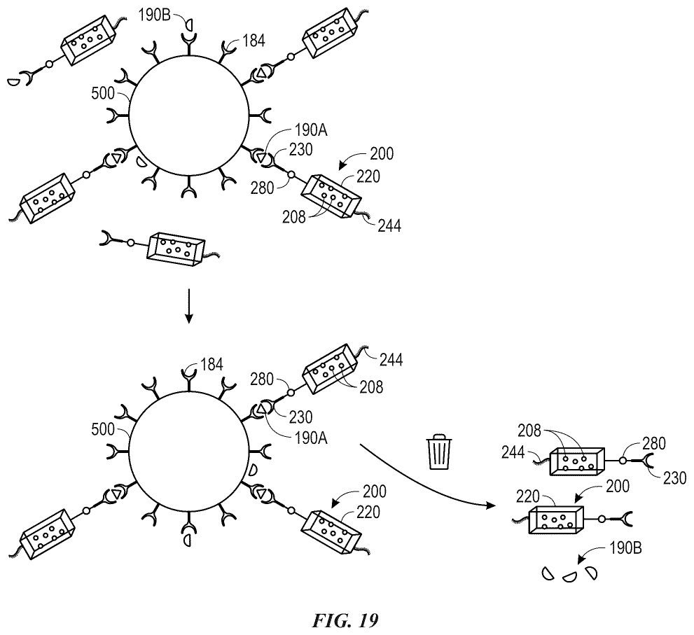

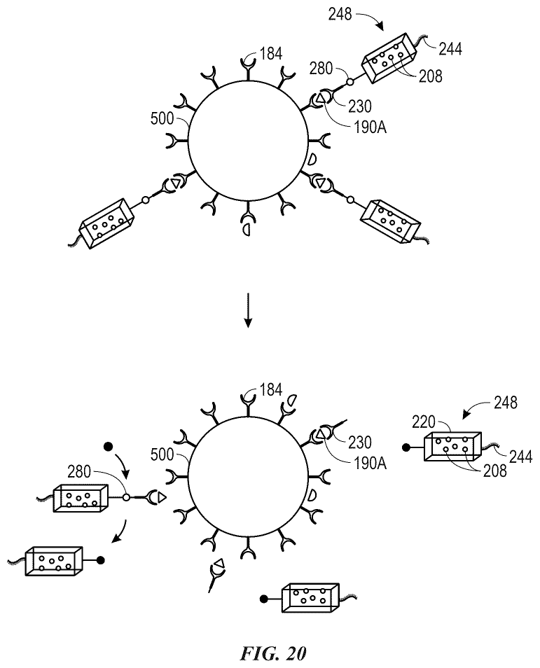

This is further illustrated in FIG. 18, in which the topmost illustration illustrates the protein+bead/binder complex 454 formed by the analyte of interest 190 and the localization affinity binders 184 attached to the beads 500 prior to addition of the detection supramolecular structures 200 (top) and after addition of the detection supramolecular structures 200 (bottom). As shown in this illustration, analytes of interest 190A bound to the localization affinity binders 184 at this stage are also bound at different regions or domains to the detection supramolecular structures 200, such as via a different and distinct specific interaction. As shown in FIG. 19 (bottom) unbound detection supramolecular structures 200 and analytes (e.g., analytes not of interest 190B) maybe separated from the protein+bead/binder complex 454 and bound detection supramolecular structures 200 and discarded. By way of example, the beads 500, if magnetic, may be magnetically separated from the remainder of the solution or, if not magnetic, may be spun down and the supernatant discarded.

Turning back to FIG. 17, in the detector origami implementation depicted, these outcomes are illustrated respectively by the unbound detection origami and proteins 462 that are separated from the remaining detection origami/bound protein bead complexes 466 that remain for further processing.

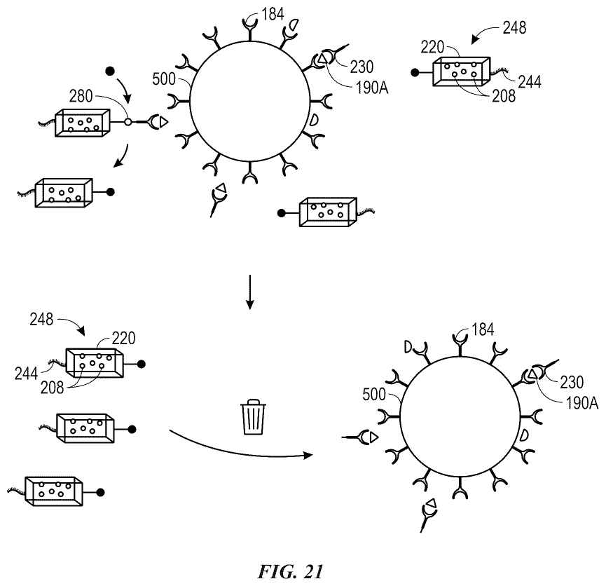

As further shown in FIG. 17, in a next step the detection origami that were successfully bound to analyte of interest at step 458, referred to as positive detection origami 140 or positive detection supramolecular structures 248 herein, are released (step 132) and subsequently isolated (step 136), aspects of which are illustrated in FIGS. 20 and 21.

By way of example, and turning to FIG. 20, the topmost illustration illustrates both localization affinity binders 184 and positive detection supramolecular structures 248 (i.e., detection supramolecular structures which have bound to a localized analyte of interest 190A) concurrently bound to the analyte of interest 190A, such as at different regions or domains. The bottom illustration of FIG. 20 illustrates a step corresponding to step 132 of FIG. 17 in which the positive detection supramolecular structures 248 are released from the analyte of interest 190A via activation or operation of the detachment site 280 present in the linkage to the affinity binder 230. As noted herein, detachment of the affinity binder 230 from the core structure 220 of the positive detection supramolecular structure 248 may be accomplished via any suitable operation including, but not limited to, photo-cleavage, chemical cleavage, or strand displacement.

As shown in FIG. 20 (bottom illustration), after displacement and release of the positive detection supramolecular structures 248, the positive detection supramolecular structures 248 are unlinked from the analyte of interest 190A, which remains bound to the bead 500. The beads 500 may then be removed from the solution magnetically (if the beads 500 are magnetic) or spun or via centrifuge as shown in FIG. 21 to separate the unbound positive detection supramolecular structures 248 from the beads 500. The positive detection supramolecular structures 248 may then be used in an assay step as discussed herein.

Turning back to the DNA origami-based protein assay example of FIG. 17, and as previously described, the isolated positive detection origami 140 of the depicted example may be flowed over or otherwise applied to a detection substrate 290 to which the positive detection origami 140 are configured to bind or otherwise attach. This is illustrated in the example of FIG. 17 at step 144 at which the positive detection origami 140 are hybridized with the detection substrate 290, such as to attachment sites 294, wells, or pads of the detection substrate 290.

As shown in the particular example of FIG. 17, once hybridized to the detection substrate 290, the positive detection origami 140 may be counted (step 148) to determine the presence and/or quantity of the analyte molecules of interest 190A in the original sample 100. As noted herein, due to the association of a single affinity binder 230 to each detection supramolecular substrate 200, a 1:1 ratio between detection supramolecular substrate 200 and molecules of analyte of interest 190A may be obtained such that counting the positive detection origami 140 is functionally equivalent to counting the individual molecules of analyte of interest 190A in the sample 100. Therefore, in this example, the result of the count may constitute an assay result 152 corresponding to the presence and/or quantity (absolute or relative) of the analyte of interest within the sample 100. Of note, at the hybridization and count steps, none of the analyte of interest 190A is present on the positive detection supramolecular structures 248 or otherwise bound to the detection substrate 290. Indeed, the analyte of interest 190A, which remains bound to the localization supramolecular structures 176 and hydrogel matrix 160, may be discarded prior to steps 144 and 168 associated with the assay. It may be noted that the absence of binding of the analyte of interest 190A, or more generally the absence of the analyte of interest 190A in the presence of the detection substrate 290 allows for a cleaner substrate with no specific binding or interaction with the analyte(s). This in turn may allow the detection substrate to be renewed and regenerated for use in subsequent assay operations.

As previously described, in practice the steps of detecting and/or counting the positive detection supramolecular structures 248 may be accomplished via the excitation and emissions of the fluorescent molecules 208 present on the detection supramolecular structures 100. Thus, the type of emission (e.g., wavelength or frequency), the intensity of emission, and so forth, may be used to detect and/or count positive detection supramolecular structures 248, each corresponding to an analyte of interest 190A. In embodiments where each positive detection supramolecular structures 248 was bound to a single analyte of interest molecule 190A and where a single positive detection supramolecular structures 248 binds to each attachment site 294, a count of emitting attachment sites can provide a count of the number of molecules of the analyte of interest present in the sample 100. Further, as described herein, in certain embodiments hybridization and counting steps may be multiplexed so as to allow multiple analytes of interest 190A to be assayed in a given operation.

While the preceding example relates one implementation employing a bead substrate to which an affinity binder is directly attached, in other contexts, as discussed in other embodiments herein, it may be useful to employ an intermediary structure to reduce or mitigate steric hinderance, and saturation, at the surface of the bead 500. By way of example, beads, such as magnetic beads, may be used as a substrate for attachment of localization supramolecular structures 176 (e.g., nucleic acid origami structures having a core origami structure with anchor molecule 180 and affinity binder 184 attached constituents) and the analyte of interest may be captured to the localization supramolecular structures 176 as opposed to the surface of the bead itself. In practice, such beads may be coated with streptavidin and/or may otherwise be conducive to attaching monoclonal antibodies or nucleic acid strands (e.g., DNA). Such an approach may prevent saturation effects due to steric hinderance and may thereby facilitate a 1:1 counting of molecules of the analyte of interest 190A.

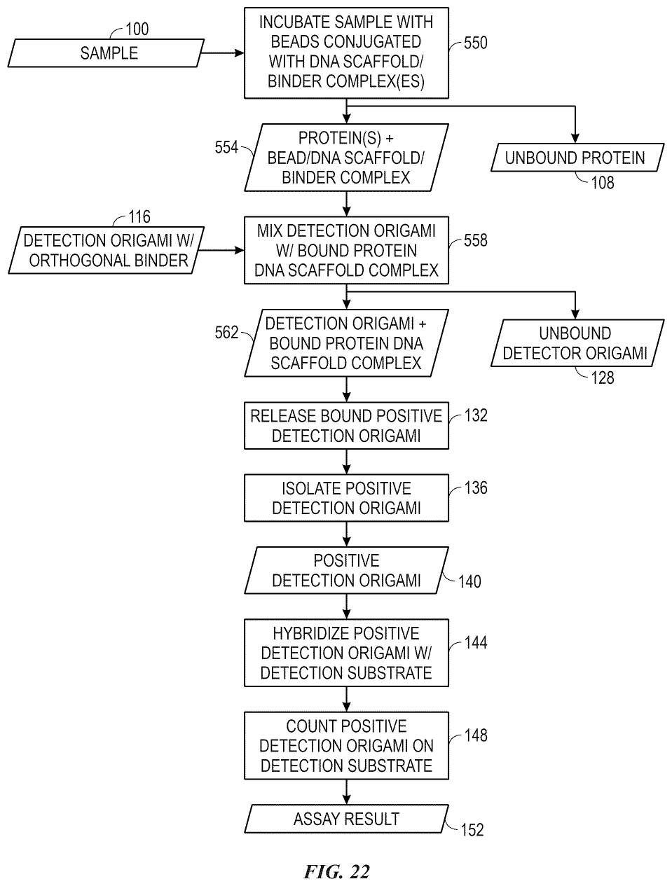

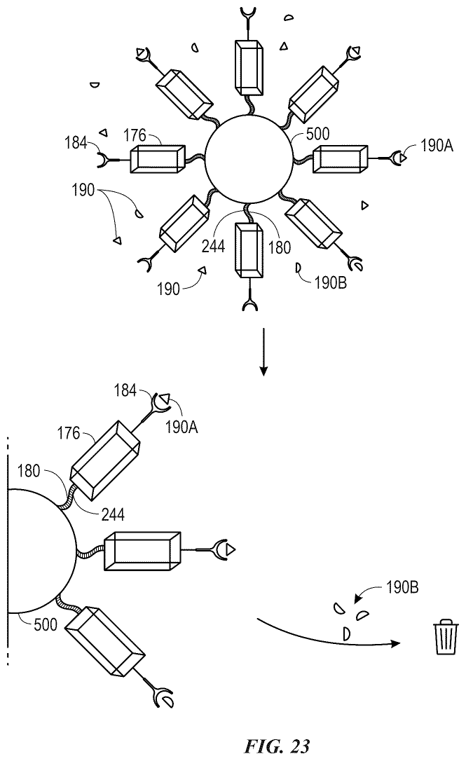

By way of example, and turning to FIG. 22 and the associated figures, in a further embodiment the affinity binders 184 used to localize the molecules of the analyte of interest 190A may be attached or otherwise linked to localization supramolecular structures 176 which themselves are attached to beads 150, such as magnetic beads. By way of illustration, and turning to FIG. 23, in some embodiments, each localization supramolecular structure 176 comprises an anchor molecule 180 linked to the core structure. In some embodiments, the anchor molecule 180 comprises an amine, a thiol, a DBCO, a maleimide, biotin, an azide, an acrydite, an NHS-ester, a single stranded nucleic acid (RNA or DNA) of specific sequence, or combinations thereof. In such embodiments, the anchor molecule 180 may facilitate binding of the localization supramolecular structure 176 to respective binding sites on a surface of a substrate, such as to an attachment site of the bead structure 500. Such bead structures 500 may comprise a plurality of binding sites, wherein each binding site is configured to link with a corresponding localization supramolecular structure 176 via the respective anchor molecule 180. By way of example, when bound to a bead 500, the localization supramolecular structure 176, and any analyte bound to the localization supramolecular structure 176 may be substantially fixed in location with respect to the bead 500. By way of example, in the depicted example, each localization supramolecular structure 176 has an anchor molecule 180 in the form of a single strand nucleic acid anchor strand that is complementary to a complement strand 244 of nucleic acid seeded on the surface of the bead 500.

As previously described, each localization supramolecular structure 176 also includes, bound to the core structure, an affinity binder 184 (or reactive group) that is specific to the analyte molecule of interest 190A, such as to a specific binding region or domain on the analyte molecule of interest 190A. As discussed herein, a respective analyte molecule may bind with specificity to the affinity binder 184 or reactive group of a localization supramolecular structure 176 through a chemical bond. In some embodiments, the affinity binder 184 or reactive group comprises a protein, a peptide, an antibody, an aptamer (e.g., RNA and DNA-based aptamers), a darpin, a polymer like PEG, or combinations thereof.

As previously noted, in certain embodiments attachment of the affinity binder 184 is via a linker structure or strand that chemically links to random or targeted (i.e., non-random) locations (e.g., complementary linker structures or strands) on one or more core molecules of the core structure of the localization supramolecular structure 176. By way of example, and in the context of affinity binders 184 (e.g., antibodies) linked to a DNA origami structure, such linkage may be accomplished using complementary nucleic acid strand pairing or other complementary pairing, via covalent or other chemical bond formation, and/or by any other suitable attachment mechanism. By way of example, linkage of affinity binders 184 may be accomplished via the mechanism of linking to specific and known “staple” strands of nucleic acid used in the formation of scaffolded nucleic acid origami structures. In such an embodiment, because the sequence of such staple strands is known and because they occur in a fixed quantity and at fixed locations on the origami structure, the affinity binder 184 may be positioned on the localization supramolecular structure 176 at a known and specific location. By way of example, certain of the staples may react with or link to a respective polymer linker with specificity, where the polymer linker corresponds to placement of the affinity binder 184. Due to the selectivity of the staples in terms of binding to specific locations on the scaffold, an affinity binder 184 may be targeted for attachment to a respective staple which selectively binds to the scaffold at a known and specific location on the localization supramolecular structure 176 (e.g., DNA origami). The resulting localization supramolecular structure 176 has binding specificity to a specific analyte molecule, as determined by the affinity binder 184, and in the presence of the specific analyte molecule will bind to one such molecule.

As shown in FIG. 22 and FIG. 23, which illustrates aspects of the step 550, a sample 100 comprising analytes 190, including analytes of interest 190A, is exposed (such as in solution) to the beads 500 having complexed localization supramolecular structures 176 in this example embodiment. All or some portion of the analyte molecules of interest 190A bind to the affinity binders 184 present on the localization supramolecular structures 176 bound to the beads 500. By way of example, the analyte molecules of interest 190A may bind via a specific interaction to the affinity binders 184 due to the specificity of the affinity binder 184 for the analyte of interest 190A. Some portion of other analyte molecules (i.e., analytes not of interest 190B) may also interact with the affinity binders 184 via non-specific interactions, but such interactions, if present will be weak and limited in number.

After an incubation period, the unbound analyte molecules 190 (e.g., analytes not of interest 190B) may be separated from the beads 500, such as via magnetic separation (if the beads 500 are magnetic) or centrifugation, as shown in the bottom step illustrated in FIG. 23. At this stage, therefore, primarily analytes of interest 190A remain bound to the bead 500 with unbound analytes having been removed or otherwise separated from the beads 500.

Turning back to FIG. 22, this is illustrated in the example, process flow in the context of a protein assay in which a nucleic acid origami forms the basis of the localization supramolecular structures 176. Hence unbound protein 108 is shown as being separated and removed subsequent to the mixing and incubation step 550, leaving the protein of interest bound to the Bead/DNA scaffold-binder complex (complex 554 in the aggregate).

In the present example embodiment, at a next stage a detection supramolecular structure 200 as discussed herein (e.g., a detection origami 116 in the process flow example of FIG. 22) is mixed (step 558) or flowed over the protein+bead/scaffold/binder complex 112, such as via mixing in solution. In the depicted example, the localization super molecular structures 176 and the detection supramolecular structures 200 have respective affinity binders 184, 230 that are specific to the analyte of interest 190A at different regions or domains (i.e., orthogonal binders). In this manner, both the localization supramolecular structure 176 and the detection supramolecular structure 200 can simultaneously bind to a respective analyte of interest 190A.

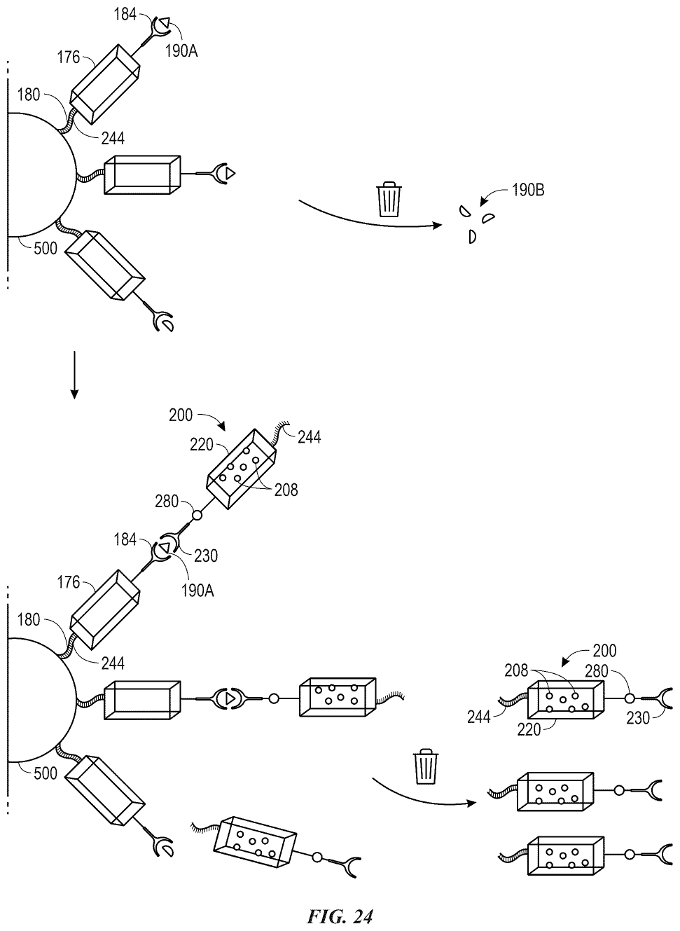

This is further illustrated in FIG. 24, in which the topmost illustration illustrates the protein+bead/scaffold/binder complex formed by the analyte of interest 190A and the localization supramolecular structures 176 prior to addition of the detection supramolecular structures 200 (top) and after addition of the detection supramolecular structures 200 (bottom). As shown in this illustration, analytes of interest 190A bound to the localization supramolecular structures 176 at this stage are also bound at different regions or domains to the detection supramolecular structures 200, such as via a different and distinct specific interaction. As also illustrated, at this stage, unbound detection supramolecular structures 200 may be separated from the beads 500, such as via magnetic separation (if the beads 500 are magnetic) or centrifugation, as shown in the bottom step illustrated in FIG. 24. Turning back to FIG. 22, in the detector origami implementation depicted, these outcomes are illustrated respectively by the unbound detection origami 128 that are washed away and the remaining detection origami/bound protein complex 562 that remain for further processing.

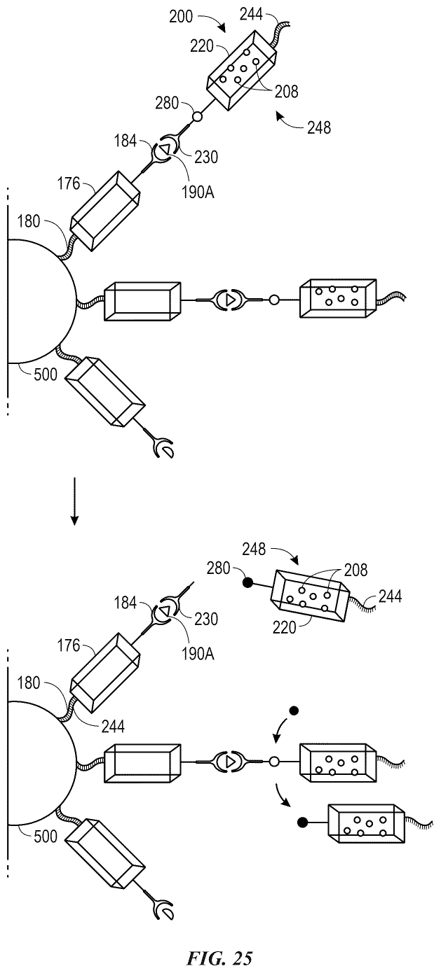

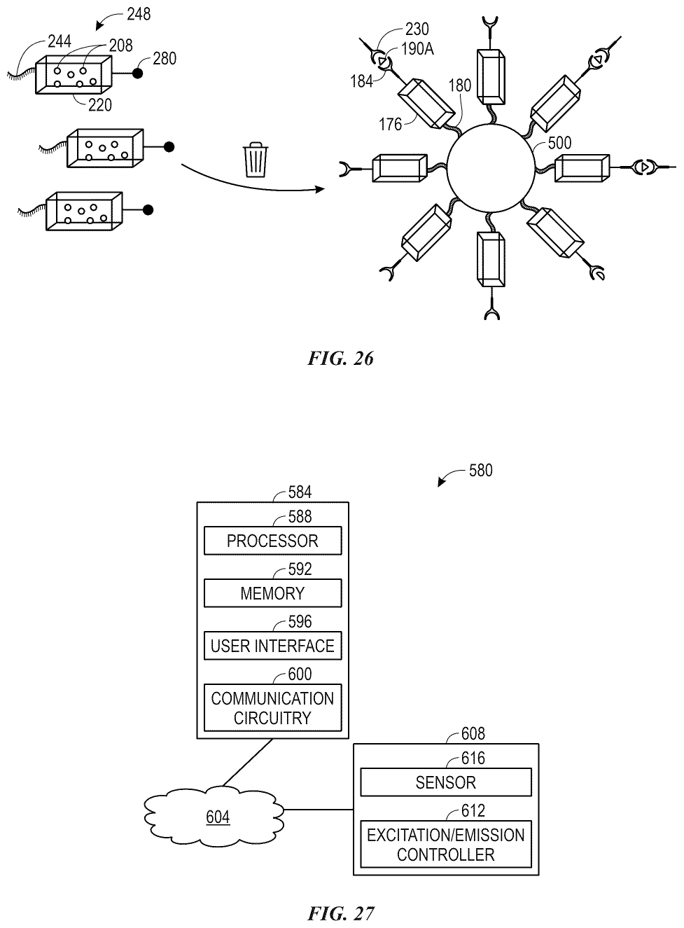

As further shown in FIG. 22, in a next step the detection origami 116 that were successfully bound to analyte of interest 190A at step 558, referred to as positive detection origami 140 or, more generally, positive detection supramolecular structures 248 herein, are released (step 132) and subsequently isolated (step 136), aspects of which are illustrated in FIGS. 25 and 26.

By way of example, and turning to FIG. 25, the topmost illustration illustrates both localization supramolecular structures 176 and positive detection supramolecular structures 248 (i.e., detection supramolecular structures which have bound to a localized analyte of interest 190A) concurrently bound to the analyte of interest 190A, such as at different regions or domains. The bottom illustration of FIG. 25 illustrates a step corresponding to step 132 of FIG. 22 in which the positive detection supramolecular structures 248 are released from the analyte of interest 190A via activation or operation of the detachment site 280 present in the linkage to the affinity binder 230. As noted herein, detachment of the affinity binder 230 from the core structure 220 of the positive detection supramolecular structure 248 may be accomplished via any suitable operation including, but not limited to, photo-cleavage, chemical cleavage, or strand displacement

As shown in FIG. 25 (bottom illustration), after displacement and release of the positive detection supramolecular structures 248, the positive detection supramolecular structures 248 are unlinked from the analyte of interest 190A, which remains bound to the bead 500 via the localization supramolecular substrates 176. The beads 500 may then be removed from the solution magnetically (if the beads 500 are magnetic) or spun or via centrifuge as shown in FIG. 26 to separate the unbound positive detection supramolecular structures 248 from the beads 500. The positive detection supramolecular structures 248 may then be used in an assay step as discussed herein.

Turning back to the DNA origami-based protein assay example of FIG. 22, and as previously described, the isolated positive detection origami 140 of the depicted example may be flowed over or otherwise applied to a detection substrate 290 to which the positive detection origami 140 are configured to bind or otherwise attach. This is illustrated in the example of FIG. 22 at step 144 at which the positive detection origami 140 are hybridized with the detection substrate 290, such as to attachment sites 294, wells, or pads of the detection substrate 290.

As shown in the particular example of FIG. 22, once hybridized to the detection substrate 290, the positive detection origami 140 may be counted (step 148) to determine the presence and/or quantity of the analyte molecules of interest 190A in the original sample 100. As noted herein, due to the association of a single affinity binder 230 to each detection supramolecular substrate 200, a 1:1 ratio between detection supramolecular substrate 200 and molecules of analyte of interest 190A may be obtained such that counting the positive detection origami 140 is functionally equivalent to counting the individual molecules of analyte of interest 190A in the sample 100. Therefore, in this example, the result of the count may constitute an assay result 152 corresponding to the presence and/or quantity (absolute or relative) of the analyte of interest within the sample 100. Of note, at the hybridization and count steps, none of the analyte of interest 190A is present on the positive detection supramolecular structures 248 or otherwise bound to the detection substrate 290. Indeed, the analyte of interest 190A, which remains bound to the localization supramolecular structures 176 and hydrogel matrix 160, may be discarded prior to steps 144 and 168 associated with the assay. It may be noted that the absence of binding of the analyte of interest 190A, or more generally the absence of the analyte of interest 190A in the presence of the detection substrate 290 allows for a cleaner substrate with no specific binding or interaction with the analyte(s). This in turn may allow the detection substrate to be renewed and regenerated for use in subsequent assay operations.

As previously described, in practice the steps of detecting and/or counting the positive detection supramolecular structures 248 may be accomplished via the excitation and emissions of the fluorescent molecules 208 present on the detection supramolecular structures 100. Thus, the type of emission (e.g., wavelength or frequency), the intensity of emission, and so forth, may be used to detect and/or count positive detection supramolecular structures 248, each corresponding to an analyte of interest 190A. In embodiments where each positive detection supramolecular structures 248 was bound to a single analyte of interest molecule 190A and where a single positive detection supramolecular structures 248 binds to each attachment site 294, a count of emitting attachment sites can provide a count of the number of molecules of the analyte of interest present in the sample 100. Further, as described herein, in certain embodiments hybridization and counting steps may be multiplexed so as to allow multiple analytes of interest 190A to be assayed in a given operation.

As may be appreciated, aspects of the presently described structures and techniques may be implemented or used if the further context of a device or system, such as an analyte detection or quantification system 580 as shown in FIG. 27. In particular, FIG. 27 shows an analyte detection or quantification system 580 that includes a controller 584. The controller 584 includes processor 588 and a memory 592 storing instructions configured to be executed by the processor 588. The controller 584 includes a user interface 596 and communication circuitry 600, e.g., to facilitate communication over the internet 604 and/or over a wireless or wired network. The user interface 596 facilitates user interaction with operational results or parameter specification as provided herein.

The processor 588 is programmed to receive data and execute operational commands for performing one or more operations as described herein, such as an analyte molecule identification and/or quantification operation using a fluorescence-based imaging system or flow cytometer. With this in mind, the system 580 also includes a detection component 608, such as an area imaging component and/or flow cytometer that operates to control operations on or involving detection supramolecular structures 200 as may be used to identify or count analyte molecules as discussed herein. An excitation and/or emission readout controller 612 may be present that controls excitation operations and/or emission readout operations performed on detection supramolecular structures 200, and so forth at appropriate time points during a detection operation. A sensor 616 may be provided as one or more of an optical sensor (e.g., a fluorescent sensor, an infrared sensor), an image sensor, an electrical sensor, or a magnetic sensor for detecting suitable data generated by or at the detection supramolecular structures 200.

### Data and Examples

With the preceding examples and implementations in mind, data and illustrations generated in developing the presently disclosed techniques is provided. By way of example, and with reference to the implementation illustrated and discussed with respect to FIG. 9A, certain illustrative data is described herein. In particular, and as described herein, FIG. 9A relates to an implementation of hybridization and counting steps that may be multiplexed so as to allow multiple analytes of interest 190A to be assayed in a given operation. In such an example, as part of processing a given sample 100 different sets of localization supramolecular structures 176 and detection supramolecular structures 200 may be employed wherein each set has respective affinity binders 184, 230 for different analytes 190. In certain embodiments the respective positive detection supramolecular structures 248 may be distinguished during the assay operation based upon different fluorophores 208 or by different ratios or combinations of fluorophores 208. As discussed in the referenced example illustrated in FIG. 9A, positive detection supramolecular structures 248A, 248B, and 248C may each have respective binding specificities to different analytes of interest. Correspondingly, the positive detection supramolecular structures 248A 248B, and 248C may be characterized by different attached fluorophores 208A, 208B, and 208C respectively which differ in their excitation wavelength response and/or emission wavelengths, thereby allowing the respective positive detection supramolecular structures 248 to be differentiated at the detection substrate 290 (e.g., a chip device having binding or reaction sites defined on the surface of the chip.

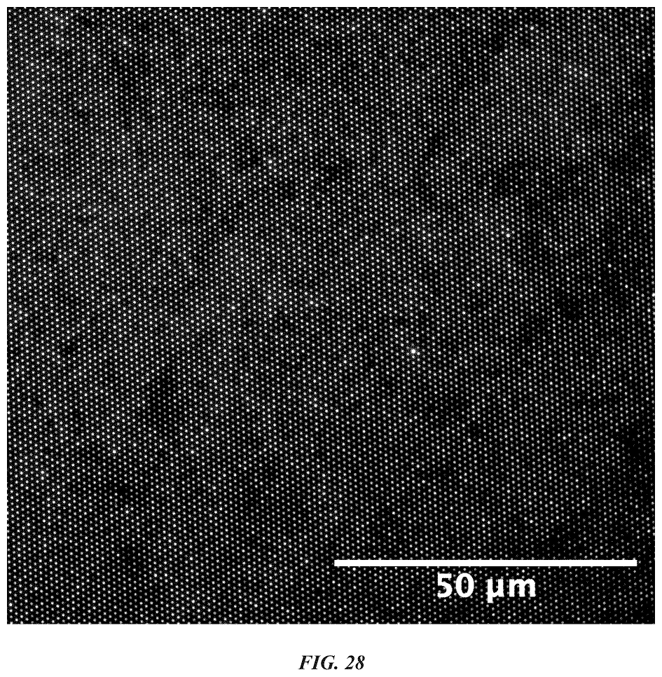

Turning to FIG. 28, a proof-of-concept study was performed using a configuration corresponding to that shown in FIG. 9A in order to evaluate the performance of a metafluorophore reporter in the context of chip substrates. In this study the chip substrates were treated with ammonium persulfate (APS) to improve the properties of the binding sites as hosts for metafluorophore box DNA origami structures (e.g., positive detection supramolecular structures 248).

As illustrated in FIG. 28, depicting a plan or top-down view of a chip substrate (e.g., detection substrate 290) having binding sites 294, metafluorophore box DNA origami structures were successfully bound to the binding sites and the interstitial regions had negligible amounts of background fluorescence. In this example, a large fraction of binding sites (in the depicted example, greater than 90%) were demonstrated to be occupied by the metafluorophore box DNA origami structures. A number of experiments were conducted where the metafluorophore box DNA origami structures were loaded with varying number of fluorophores. While the observed intensity at the binding sites was proportional to the number of fluorescent dyes being hosted by specific metafluorophore box DNA origami structures species, the fraction of binding sites on the chip surfaces covered by the DNA origami boxes remained invariant, within reasonable experimental variation, for boxes with varying number of fluorescent dyes.

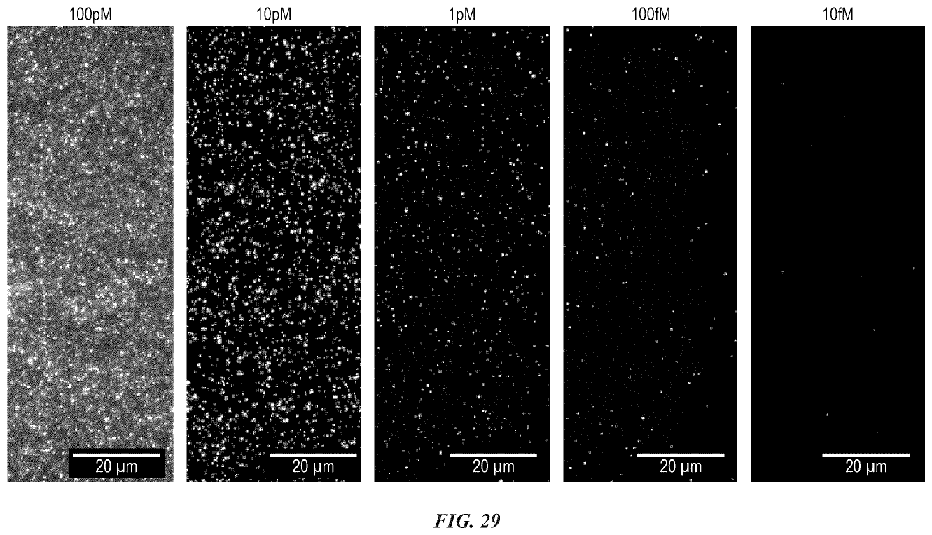

To further validate the proposed techniques, the proposed quantification pipeline was challenged using a variable concentration of DNA origami metafluorophores with each box origami structure designed to contain the same number of fluorescent dyes. As seen in FIG. 29, the occupancy of binding sites 294 on the chip substrate surface was observed to be directly proportional to the concentration (e.g., 100 pM, 10 pM, 1 pM, 100 fM, and 10 fM) of metafluorophore box DNA origami structures incubated with the chip substrate.

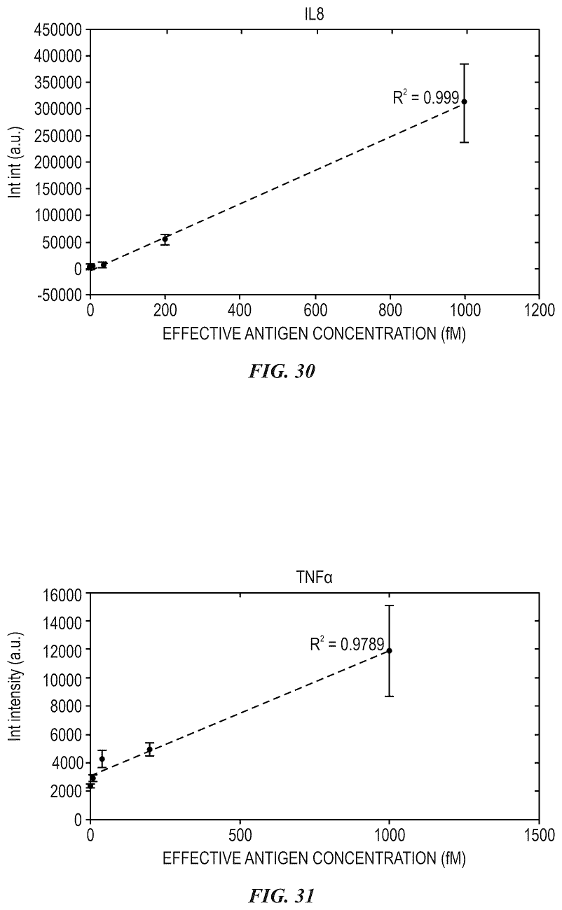

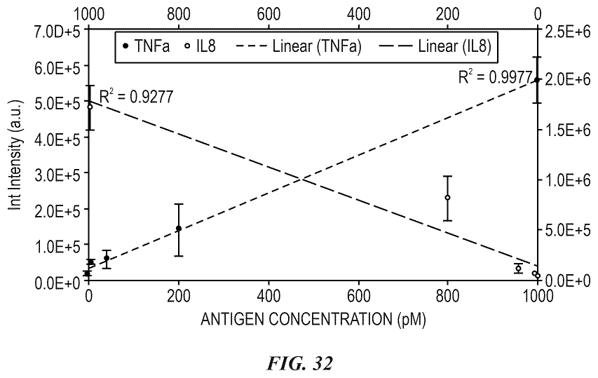

In addition, turning to FIGS. 30, 31, and 32, examples of the quantification of the output of an actual assay are graphically illustrated. In these examples, FIGS. 30 and 31 demonstrate the quantification of respective assays configured to quantify the concentration of interleukin-8 (IL8) and tumor necrosis factor alpha (TNFα). As illustrated in FIGS. 30 and 31, the positive correlation between protein concentration and the fluorescent signal measured from the binding sites on the chip substrates is observable. Turning to FIG. 32, in this figure the ability to successfully quantify protein concentrations in a multiplexed format is demonstrated. In particular, in this example the assay was performed with the concentrations of TNFα and IL8 having opposing concentration trends (i.e., as one is increased the other is decreased). Using metafluorophore box DNA origami structures as described in the techniques herein, the concentration of both protein analytes could be measured with high fidelity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An analyte quantification system, comprising:
a detection component comprising:
an excitation and emission controller configured to stimulate emissions by one or more detection supramolecular structures when present in a detection area;
one or more sensors configured to detect emissions from the one or more detection supramolecular structures when present in the detection area;
a controller in communication with the detection component, wherein the controller is configured to quantify one or more analytes of interest based on detected emissions from the one or more detection supramolecular structures within the detection area and wherein analytes of interest are not present in the detection area when emissions are detected, wherein each detection supramolecular structure has an affinity for a single analyte of interest and is configured to bind to only a single copy of the analyte to which it has affinity.

2. The analyte quantification system of claim 1, wherein the detection component comprises a flow cytometer and the detection area comprises a flow tube of the flow cytometer.

3. The analyte quantification system of claim 1, wherein the detection component comprises an area imager configured to image a substrate on which the one or more detection supramolecular structures are bound via complementary nucleic acid hybridization or via bridging salt molecules.

4. The analyte quantification system of claim 1, wherein the controller is configured to quantify the one or more analytes of interest based on detected emissions from detection supramolecular structures previously bound to and released from respective analyte molecules of interest prior to the detection supramolecular structures being introduced to the detection area.

5. A detection supramolecular structure, comprising:
a nucleic acid supramolecular structure;
an affinity binder linker comprising a detachment site;
an affinity binder linked to the affinity binder linker and having a binding affinity for a specific analyte;
a plurality of florescent molecule linkers; and
a plurality of fluorescent molecules, wherein each fluorescent molecule is linked to a respective fluorescent molecule linker, wherein the plurality of fluorescent molecules is indicative of the affinity binder linked to the detection supramolecular structure.

6. The detection supramolecular structure of claim 5, wherein the nucleic acid supramolecular structure comprises a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, enzymatically synthesized nucleic acid structures, or structures created by nucleic acid tile assembly.

7. The detection supramolecular structure of claim 5, wherein the nucleic acid supramolecular structure is scaffolded with one or more scaffolds.

8. The detection supramolecular structure of claim 5, wherein the nucleic acid supramolecular structure comprises a prescribed two-dimensional (2D) or three-dimensional (3D) shape.

9. The detection supramolecular structure of claim 5, wherein the specific analyte comprises a specific protein.

10. The detection supramolecular structure of claim 5 further comprising an anchor molecule configured to immobilize the detection supramolecular structure on a detection substrate.

11. The detection supramolecular structure of claim 5, wherein the fluorescent molecules comprise dye molecules or quantum dots.

12. The detection supramolecular structure of claim 5, wherein the affinity binder comprises one or more of a protein, a peptide, an antibody, an aptamer, a darpin, or a polymer.

13. The detection supramolecular structure of claim 5, wherein the detachment site comprises one or more of a photo-cleavage site, a chemical cleavage site, or a strand displacement site.

14. A detection supramolecular structure, comprising:
a nucleic acid supramolecular structure;
an affinity binder linker comprising a detachment site;
an affinity binder linked to the affinity binder linker and having a binding affinity for a specific analyte;
a rolling circle amplification (RCA) tag; and
an RCA template associated with the RCA tag and comprising a plurality of copies of two or more sequences, wherein the two or more sequences, in combination, are indicative of the affinity binder linked to the detection supramolecular structure.

15. The detection supramolecular structure of claim 14, wherein the nucleic acid supramolecular structure comprises a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, enzymatically synthesized nucleic acid structures, or structures created by nucleic acid tile assembly.

16. The detection supramolecular structure of claim 14, wherein the nucleic acid supramolecular structure is scaffolded with one or more scaffolds.

17. The detection supramolecular structure of claim 14, wherein the nucleic acid supramolecular structure comprises a prescribed two-dimensional (2D) or three-dimensional (3D) shape.

18. The detection supramolecular structure of claim 14, wherein the specific analyte comprises a specific protein.

19. The detection supramolecular structure of claim 14, further comprising an anchor molecule configured to immobilize the detection supramolecular structure on a substrate.

20. The detection supramolecular structure of claim 14, wherein the plurality of copies of the sequences taken together comprise a characteristic ratio of each sequence that is indicative of the affinity binder linked to the detection supramolecular structure.

21. The detection supramolecular structure of claim 14, wherein the detachment site comprises one or more of a photo-cleavage site, a chemical cleavage site, or a strand displacement site.

22. The detection supramolecular structure of claim 14, further comprising a nanoball formed as an amplicon of the RCA template formed as a continuous strand comprising a repeated and sequential sequence of the plurality of copies of the two or more sequences.

23. The detection supramolecular structure of claim 22, further comprising a respective and corresponding fluorophore bound by complementary pairing to each copy of a respective sequence of the nanoball.

24. A detection supramolecular structure, comprising:
a nucleic acid supramolecular structure;
an affinity binder linker comprising a detachment site;
an affinity binder linked to the affinity binder linker and having a binding affinity for a specific analyte;
a decode tag comprising a plurality of copies of two or more sequences, wherein the two or more sequences, in combination, are indicative of the affinity binder linked to the detection supramolecular structure.

25. The detection supramolecular structure of claim 24, wherein the nucleic acid supramolecular structure comprises a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, enzymatically synthesized nucleic acid structures, or structures created by nucleic acid tile assembly.

26. The detection supramolecular structure of claim 24, wherein the nucleic acid supramolecular structure is scaffolded with one or more scaffolds.

27. The detection supramolecular structure of claim 24, wherein the nucleic acid supramolecular structure comprises a prescribed two-dimensional (2D) or three-dimensional (3D) shape.

28. The detection supramolecular structure of claim 24, wherein the specific analyte comprises a specific protein.

29. The detection supramolecular structure of claim 24, further comprising an anchor molecule configured to immobilize the detection supramolecular structure on a substrate.

30. The detection supramolecular structure of claim 24, wherein the plurality of copies of the sequences taken together comprise a characteristic ratio of each sequence that is indicative of the affinity binder linked to the detection supramolecular structure.

31. The detection supramolecular structure of claim 24, wherein the detachment site comprises one or more of a photo-cleavage site, a chemical cleavage site, or a strand displacement site.

32. A method for forming a detection supramolecular structure, the method comprising the steps of:
synthesizing or acquiring a nucleic acid supramolecular structure;
attaching an affinity binder linker comprising a detachment site to the nucleic acid supramolecular structure;
attaching an affinity binder to the affinity binder linker, wherein the affinity binder has a binding affinity for a specific analyte, wherein the detachment site on the affinity binder linker is configured to detach the affinity binder from the nucleic acid supramolecular structure when the detachment site is activated; and
attaching a plurality of florescent molecule linkers to the nucleic acid supramolecular structure; and
attaching a fluorescent molecule to each fluorescent molecule linker, wherein the plurality of fluorescent molecules is indicative of the affinity binder linked to the detection supramolecular structure.

33. The method of claim 32, further comprising:
attaching an anchor molecule to the nucleic acid supramolecular structure, wherein the anchor molecule is configured to immobilize the detection supramolecular structure on a detection substrate.

34. The method of claim 32, wherein the fluorescent molecules comprise dye molecules or quantum dots.

35. The method of claim 32, wherein the affinity binder comprises one or more of a protein, a peptide, an antibody, an aptamer, a darpin, or a polymer.

36. The method of claim 32, wherein the detachment site comprises one or more of a photo-cleavage site, a chemical cleavage site, or a strand displacement site.

37. A method for quantifying an analyte of interest in a sample, the method comprising:
exposing a plurality of detection supramolecular structures to one or more molecules of an analyte of interest, wherein each molecule of the analyte of interest is bound to a respective localization affinity binder, wherein each detection supramolecular structure comprises a detection affinity binder configured to bind to a respective molecule of the analyte of interest at a different location than the respective localization affinity binder;
separating and removing detection supramolecular structures that have not bound to respective molecules of the analyte of interest;
activating a detachment site of an affinity binder linker of each detection supramolecular structure that has bound to respective molecules of the analyte of interest to separate the respective detection affinity binder from the respective detection supramolecular structure to generate one or more positive detection supramolecular structures;
separating the positive detection supramolecular structures from the molecules of the analyte of interest; and
generating an assay result based on detection of the positive detection supramolecular structures.

38. The method of claim 37, wherein exposing the plurality of detection supramolecular structures to one or more molecules of the analyte of interest comprises flowing the plurality of detection supramolecular structures through a hydrogel matrix on which the localization affinity binders are attached.

39. The method of claim 37, wherein exposing the plurality of detection supramolecular structures to one or more molecules of the analyte of interest comprises flowing the plurality of detection supramolecular structures through a hydrogel matrix on which a plurality of localization supramolecular structures is attached, wherein each localization supramolecular structure comprises a respective localization affinity binder.

40. The method of claim 37, wherein exposing the plurality of detection supramolecular structures to one or more molecules of the analyte of interest comprises mixing the plurality of detection supramolecular structures in solution with a plurality of beads on which the localization affinity binders are attached.

41. The method of claim 37, wherein exposing the plurality of detection supramolecular structures to one or more molecules of the analyte of interest comprises mixing the plurality of detection supramolecular structures in solution with a plurality of beads on which a plurality of localization supramolecular structures is attached, wherein each localization supramolecular structure comprises a respective localization affinity binder.

42. The method of claim 37, wherein activating the detachment site comprises performing one or more of a photo-cleaving operation, a chemical cleaving operation, or a strand displacement operation.

43. The method of claim 37, wherein separating the positive detection supramolecular structures from the molecules of the analyte of interest comprises flowing the positive detection supramolecular structures through a hydrogel matrix.

44. The method of claim 37, wherein separating the positive detection supramolecular structures from the molecules of the analyte of interest comprises separating beads on which the molecules of the analyte of interest are directly or indirectly bound from the positive detection supramolecular structures using magnetic or centrifugal forces.

45. The method of claim 37, wherein generating the assay result comprises counting the positive detection supramolecular structures.

47. The method of claim 37, wherein generating the assay result comprises:
hybridizing the positive detection supramolecular structures to a detection substrate; and
counting the positive detection supramolecular structures on the detection substrate.

48. The method of claim 37, wherein generating the assay result comprises:
associating the positive detection supramolecular structures to attachment sites of a detection substrate via non-specific interactions mediated by the presence of bridging salt molecules; and
counting the positive detection supramolecular structures on the detection substrate.

49. The method of claim 48, wherein the bridging salt molecules comprise ammonium persulfate.

50. The method of claim 37, wherein each detection supramolecular structure comprises:
a nucleic acid supramolecular structure;
the affinity binder linker comprising the detachment site; and
the detection affinity binder linked to the affinity binder linker and having a binding affinity for the analyte of interest.

51. The method of claim 48, further comprising:
a plurality of florescent molecule linkers; and
a plurality of fluorescent molecules, wherein each fluorescent molecule is linked to a respective fluorescent molecule linker, wherein the plurality of fluorescent molecules is indicative of the detection affinity binder linked to the detection supramolecular structure.
